(19) United States
(12) Patent Application Publication
(10) Pub. No.: US 2019/0282094 A1
(43) Pub. Date: Sep. 19, 2019

(54) Wireless Smart Contact Lens for Intraocular Pressure Measurement

(71) Applicant: Menicon Co. Ltd., Nagoya (JP)

(72) Inventors: Mouad Lamrani, Geneva (CH); Sami Antero Lakka, Lempäälä (FI)

(21) Appl. No.: 16/351,345

(22) Filed: Mar. 12, 2019

Related U.S. Application Data
(60) Provisional application No. 62/642,913, filed on Mar. 14, 2018.
(60) Provisional application No. 62/642,926, filed on Mar. 14, 2018.

(51) Int. Cl. A61B 3/16; G02C 7/04

(52) U.S. Cl. CPC A61B 3/16; G02C 7/04

(57) ABSTRACT

A contact lens includes a lens material and a sensor coupled to the lens material. The sensor is configured to sense a measurable characteristic used to determine an intraocular pressure of an eye to which the contact lens is applied. The sensor can include a variable capacitance sensor configured to change capacitance in response to a mechanical strain of the lens material. The intraocular pressure of the eye can then be correlated to the mechanical strain of the lens material.

### CROSS-REFERENCE TO RELATED APPLICATIONS

This application claims priority from U.S. Provisional Application No. 62/642,913 filed on Mar. 14, 2018, the disclosure of which is incorporated herein, in its entirety, by this reference. This application also claims priority from U.S. Provisional Application No. 62/642,926 filed on Mar. 14, 2018, the disclosure of which is incorporated herein, in its entirety, by this reference.

### FIELD

The present disclosure relates to the field of contact lenses, particularly to contact lenses capable of wirelessly monitoring an intraocular pressure of an eye.

### BACKGROUND

Glaucoma is a leading cause of blindness worldwide and, although it is more common in adults over age 35, it can occur at any age. Glaucoma primarily arises when the intraocular pressure of the eye increases to unhealthy levels. The fluid responsible for pressure in the eye is the aqueous humor. It is a transparent fluid produced by the eye in the ciliary body and collected and drained by a series of channels (trabecular meshwork, Schlemm's canal and venous system). The basic disorder in most glaucoma patients is caused by an obstruction or interference that restricts the flow of aqueous humor out of the eye and thereby increases pressure within the eye.

Increased pressure within the eye can cause progressive damage to the optic nerve. As optic nerve damage occurs, characteristic defects in the visual field can develop, which can lead to blindness if the disease remains undetected and untreated. Because of the insidious nature of glaucoma and the gradual and painless loss of vision associated therewith, glaucoma does not produce symptoms that would motivate an individual to seek help until relatively late in its course, when irreversible damage has already occurred. As a result, millions of glaucoma victims are unaware that they have the disease and face eventual blindness. Glaucoma can be detected and evaluated by measuring the eye's fluid pressure using a tonometer.

There are several methods and devices available for measuring intraocular pressure, outflow facility, and/or various other glaucoma-related characteristics of the eye. The following U.S. issued patents disclose various examples of such conventional devices and methods, and are hereby incorporated by reference for all they disclose:

One technique for measuring absolute intraocular pressure is known as ocular tonometry, and involves the use of a device known as a tonometer. There are several different types of tonometry, including applanation tonometry, indentation tonometry, air puff tonometry, and rebound tonometry, and each can use a different form of tonometer adapted to measure the absolute intraocular pressure of the eye.

For example, in applanation tonometry, eye pressure is measured by depressing or flattening the surface of the eye, and then estimating the amount of force necessary to produce the given flattening or depression. Conventional tonometry techniques using the principle of applanation can provide accurate measurements of intraocular pressure, but are subject to many errors in the way they are currently being performed. In addition, the present devices either require professional assistance for their use or are too complicated, expensive, or inaccurate for individuals to use at home. As a result, individuals must visit an eye care professional in order to check and monitor eye pressure. The frequent self-checking of intraocular pressure is useful not only for monitoring therapy and self-checking for patients with glaucoma, but also for the early detection of rises in pressure in individuals without glaucoma and for whom the elevated pressure was not detected during their office visit.

By contrast, according to indentation tonometry (Schiotz), a known weight (or force) is applied against the cornea and the intraocular pressure is estimated by measuring the linear displacement which results during deformation or indentation of the cornea. The linear displacement caused by the force is indicative of intraocular pressure. In particular, for standard forces and standard dimensions of the indenting device, there are known tables which correlate the linear displacement and intraocular pressure.

Conventional measurement techniques using applanation and indentation are subject to many errors. The most frequently used technique in the clinical setting is contact applanation using Goldman tonometers. The main sources of errors associated with this method include the addition of extraneous pressure on the cornea by the examiner, squeezing of the eyelids or excessive widening of the lid fissure by the patient due to the discomfort caused by the tonometer probe resting upon the eye, and inadequate or excessive amount of dye (fluorescein). In addition, conventional techniques typically depend upon operator skill and require that the operator subjectively determine alignment, angle, and amount of depression. Thus, variability and inconsistency associated with less valid measurements are problems encountered using conventional methods and devices for intraocular pressure measurement.

Another method of tonometry is known as rebound tonometry. This type of tonometry is often performed by a doctor who holds the end of a hand-held device proximate the user's eye. When activated, the rebound tonometer bounces a small probe, typically a plastic tipped metal probe, against the cornea of the eye. These devices commonly use an induction coil to launch the probe against the cornea. As the probe rebounds off of the cornea and retracts back into the device the induction current is measured. This current corresponds to the rate of deceleration of the probe, which is then used to calculate the absolute intraocular pressure of the eye. Although these types of devices are relatively simple to use, they nevertheless require a trained professional to obtain an intraocular pressure reading from a patient. Thus, absolute intraocular pressure readings are only taken during optometrist visits for most patients, providing only a small data set for analysis.

A variety of tonometry methods using special contact lenses have also been devised. However, such systems suffer from a number of restrictions and virtually none of these devices are being widely utilized, and are generally not accepted in the clinical setting due to their limitations and inaccurate readings. Moreover, such devices typically include instrumented contact lenses which can be cumbersome and complex to use. These complicated devices can employ expensive electronic circuitry and/or transducers embedded in the contact lens. In addition, some contact lens devices use a piezoelectric material incorporated into the lens. However, metallization of the components of the lens overlying the optical axis decreases the visual acuity of patients using this type of lens. Moreover, accuracy can be variable since the piezoelectric material is affected by small changes in temperature and the velocity with which the force is applied. There are also contact lens tonometers which utilize fluid in a chamber to cause the deformation of the cornea; however, the fluid therein has a tendency to accumulate in the lower portion of the chamber, thus failing to produce a stable flat surface which is necessary for an accurate measurement.

Other prior art methods use a coil wound about the inner surface of the contact lens and a magnet subjected to an externally created magnetic field. A membrane with a conductive coating is compressed against a contact, completing a short circuit. The magnetic field forces the magnet against the eye, and the force necessary to separate the magnet from the contact is considered proportional to the pressure. This type of device suffers from many limitations and drawbacks. For example, this design utilizes a current in the lens which, in turn, is in direct contact with the body, presenting a possible risk of damaging the eye with the current. These devices also lack an alignment system, and can include complex circuitry which is difficult to manufacture and can obscure a user's vision. Another similar tonometry method involves a contact lens including a cylindrical actuator positioned on the contact lens, for example as described in U.S. Pat. No. 8,914,089. Such a tonometer can include a moveable magnetic central piston which slides through a wire coil positioned on the lens. Electric current is run through the wire coil in a progressively increasing manner, which causes a force to be applied via the magnetic piston. However, these types of tonometers require circuitry and electrical power to be fed to the contact lens on the eye, greatly increasing cost and complexity. The complicated tonometer mechanism can also be bulky and lead to discomfort while the lens is on the eye. Further, the tonometer can obscure a user's vision while taking a reading.

There is a need for real time absolute intraocular pressure data because absolute intraocular pressure readings can vary, and it is often desirable to have a large data set when patients and their health care providers make eye care or contact lens decisions. The ability for patients to take frequent absolute intraocular pressure measurements without the need for a third party would thus be greatly beneficial. In order for large amounts a data regarding absolute intraocular pressure to be gathered, it is most beneficial for readings to be taken frequently throughout the day, for example as a time series. Accordingly, a contact lens including a tonometer system should be comfortable and unobtrusive enough that a patient or user will wear the lens as they would a traditional contact lens.

### SUMMARY

In one embodiment, a contact lens includes a lens material and a sensor coupled to the lens material. The sensor is configured to sense a measurable characteristic used to determine an intraocular pressure of an eye to which the contact lens is applied.

The sensor can include a variable capacitance sensor configured to change capacitance in response to a mechanical strain of the lens material. The intraocular pressure of the eye can be correlated to the mechanical strain of the lens material.

The contact lens can include an outer surface and the sensor can include a layer of a conductive material disposed over the outer surface of the contact lens. The sensor can also include a layer of a dielectric material overlying the conductive material. The conductive material can be a transparent polymer material including poly(3,4-ethylenedioxythiophene) polystyrene sulfonate. The layer of the conductive material can include a continuous layer. The layer of the conductive material can have a thickness of from about 1 micrometer to about 10 micrometers. The dielectric material can include a polymer material. The dielectric material can include a silicone material. The dielectric material can include polydimethylsiloxane. The layer of the dielectric material can be continuous. The layer of the conductive material has a thickness of from about 1 micrometer to about 10 micrometers.

The sensor can include a second layer of the conductive material overlying the layer of the dielectric material. The sensor can include a parallel plate capacitor. A capacitance of the parallel plate capacitor can correspond to the mechanical strain of the lens material. The contact lens can further include an antenna structure forming a part of the sensor. The second conductive material can include the antenna structure. The sensor can include an electrical oscillator having a frequency corresponding to the strain of the lens material. The oscillator can be configured in series or in parallel. In the case of a series electrical oscillator, the frequency of the electrical oscillator can correspond to a factor of how much the contact lens has stretched from an initial state on an eye according to the equation:

$$f = \frac{1}{2 π {λ}^{2} \sqrt{{LC}_{0}}}$$

wherein f is the frequency of the electrical oscillator, λ is the factor of how much the contact lens has stretched on the eye, L is an inductance of the antenna structure, and C₀ is a capacitance of the capacitor when the contact lens is in the initial state. The mechanical strain of the contact lens can be proportional to λ, and a relative intraocular pressure of the eye can be proportional to λ.

The sensor can include a variable capacitance sensor having a capacitor including a first layer of a conductive material, a second layer of the conductive material, and a layer of a dielectric disposed between the first and second layers of conductive material. The sensor can also include an antenna having an inductance and electrically connected to the capacitor, the second layer of the conductive material including the antenna.

The sensor can have a thickness of from about 20 micrometers to about 50 micrometers.

The lens material can include an outer surface and the contact lens can further include an encapsulation layer. The sensor can be positioned between the lens material and the encapsulation layer. The sensor can include a first layer of dielectric material disposed over the outer surface of the lens material, a first layer of conductive material disposed over the first layer of the dielectric material, and a micromagnet configured to exert a force on at least the first layer of dielectric material and the first layer of conductive material responsive to actuation, the force exerted by the micromagnet on at least the first layer of dielectric material and the first layer of conductive material responsive to actuation configured to applanate or indent a portion of the eye. The sensor can include an application specific integrated circuit configured to measure capacitance of the sensor as the micromagnet exerts the force on at least the first layer of dielectric material and the first layer of conductive material. The capacitance can be used to determine a frequency corresponding to the strain of the lens material, the frequency being approximately 4.1395 GHz.

The sensor can include a second layer of dielectric material disposed over the first layer of conductive material, and a second layer of conductive material disposed over the second layer of the dielectric material. The micromagnet can be configured to exert the force on the eye and the variable capacitance.

The sensor can include a second layer of dielectric material disposed over the first layer of conductive material. The sensor can include a second layer of conductive material disposed over the second layer of the dielectric material. The sensor can include an application specific integrated circuit configured to measure capacitance of the sensor as the micromagnet exerts the force on at least the first layer of dielectric material, the first layer of conductive material, and the second layer of dielectric material. The micromagnet can be configured to, responsive to actuation from a magnetic actuator, exert the force on at least the first layer of dielectric material, the first layer of conductive material responsive to actuation, and the second layer of the dielectric material.

A tonometer system can be formed in the contact lens and including the sensor, and a test body in contact with the lens material and exert a force on an eye in the presence of a magnetic field when the contact lens is positioned on the eye.

The test body can include an expandable material with the sensor in contact with the lens material. The force exerted on the eye can cause a mechanical strain in the contact lens. The sensor can detect a rate of change of the mechanical strain of the contact lens displacement when the force is no longer exerted on the eye. The rate of change of the mechanical strain of the contact lens upon release of the force of the expandable material can correspond to an absolute intraocular pressure of the eye. The test body can include at least one of an expandable polymer material, an expandable magnetoresponsive hydrogel material, or an expandable magnetoresponsive elastomer material. The expandable material can expand by the alignment of polymeric magnetic microchains in the material. The expandable material can be disposed within the lens material outside an optic zone of the contact lens. The test body can be disposed on an outer surface of the contact lens outside an optic zone of the contact lens. The test body can have a diameter of from about 1 millimeters to about 3 millimeters when not in the presence of a magnetic field. The test body can have a thickness of from about 50 micrometers to about 100 micrometers. The sensor can include a parallel plate capacitor including a first layer and a second layer of a conductive material and a layer of a dielectric material disposed therebetween. The sensor can include an antenna structure.

The test body can include a micromagnet disc or a micromagnet array configured to exert the force on the eye. The sensor can detect a rate of change of the mechanical strain of the contact lens when the force is no longer exerted on the eye, the rate of change of the mechanical strain of the contact lens upon release of the force of the expandable material corresponding to an absolute intraocular pressure of the eye. The rate of change is measured by a gradiometer or the oscillation of the test body. The micromagnet disc or micromagnet array can be responsive to a magnetic actuator or any other external actuator to exert the force on the eye. The test body can be coated with a contrast agent, the contrast agent including at least one of a radiation source layer, a radiation reflector material, a dye, or a tag.

In another embodiment, a contact lens system for wirelessly determining an intraocular pressure of an eye of a user includes a contact lens and an electronic device. The contact lens includes a lens material having an outer surface and a sensor including one or more transparent polymer layers disposed over the outer surface of the lens material. The sensor is configured to sense measurable characteristic corresponding to the intraocular pressure of the eye of the user. The electronic device is configured to wirelessly receive the measurable characteristic sensed by the sensor.

In another embodiment, a method of manufacturing a sensor on a contact lens includes providing a contact lens having an outer surface, depositing a layer of a conductive material over the outer surface of the contact lens, and depositing a layer of a dielectric material over the conductive material to form the sensor on the contact lens. The sensor can be configured to measure a mechanical strain of the contact lens.

The method can also include depositing a second layer of the conductive material over the layer of the dielectric material. The method can also include depositing an encapsulation layer of the second layer of the conductive material. The method can also include depositing a base layer of the dielectric material directly on the outer surface of the contact lens prior to depositing the layer of the conductive material. The method can also include treating the base layer of the dielectric material to enable the conductive material to wet the base layer prior to depositing the layer of the conductive material thereon. Depositing the layer of the conductive or dielectric material can include spin casting the conductive or dielectric material on the contact lens. Depositing the layer of the conductive or dielectric material can include stamping the layer of the conductive or dielectric material on the contact lens. The layer of the conductive material and the layer of the dielectric material can be simultaneously stamped on the contact lens. Depositing the layer of the conductive or dielectric material can include stamping the layer of the conductive or dielectric material on the contact lens. The method can also include curing at least the deposited layer of conductive material, the deposited layer of dielectric material, and the contact lens.

The method can also include depositing at least a micromagnet over the layer of the dielectric material. The micromagnet can be configured to selectively exert a pressure on the eye and the variable capacitance. In another embodiment, a method of manufacturing a test body on a contact lens includes providing a mold having a shape corresponding to a shape of a contact lens, depositing a test body structure on an outer surface of the mold, and treating the test body structure or contact lens to adhere the test body structure to the contact lens.

The method can further include transferring the test body structure from the mold to a stamp and pressing the stamp against an outer surface of a contact lens to thereby transfer the test body structure from the stamp to the contact lens. Depositing the test body structure on the outer surface of the mold can include printing one or more layers making up the test body structure. Depositing the test body structure on the outer surface of the mold can include spin casting one or more layers including the test body structure onto the mold. Depositing a test body structure on an outer surface of the mold can include depositing a micromagnet disc or a micromagnet array on an outer surface of the mold.

In another embodiment, a method of wirelessly determining an intraocular pressure of an eye includes sending a wireless signal from an electronic device to a contact lens on the eye, the wireless signal having a signal frequency. The contact lens includes an outer surface and a sensor disposed on the outer surface. The method also includes receiving a response signal from the contact lens by the electronic device when the signal frequency of the wireless signal matches the natural frequency of the electrical oscillator. The response signal includes a measurable characteristic corresponding to the intraocular pressure of the eye. The method also includes determining the intraocular pressure of the user from the measurable characteristic of the response signal.

### BRIEF DESCRIPTION OF THE DRAWINGS

The accompanying drawings illustrate various embodiments of the present apparatus and are a part of the specification. The illustrated embodiments are merely examples of the present apparatus and do not limit the scope thereof.

FIG. 1A is a cross-sectional view of an example contact lens incorporating a variable capacitance sensor in accordance with the present disclosure.

FIG. 1B is a cross-sectional view of a central portion of an example contact lens incorporating a variable capacitance sensor in accordance with the present disclosure.

FIG. 1C is a cross-section view of a central portion of an example contact lens incorporating a variable capacitance sensor and a micromagnet in accordance with the present disclosure.

FIG. 2 is a top view of an example contact lens system including a contact lens incorporating a variable capacitance sensor and an antenna, and an electronic device in accordance with the present disclosure.

FIG. 3A illustrates an example circuit diagram of a variable capacitance sensor in accordance with the present disclosure.

FIG. 3B is a graph comparing measured natural frequency relative to applied pressure. Vertical axis show the last digits of a frequency around 4.139500000 GHz

FIG. 3C-3G are graphs of test data used to determine intraocular pressure using capacitance.

FIG. 3H is a cross-sectional diagram of a contact lens used to measure capacitance at various eye pressures.

FIG. 4A illustrates a cross-sectional view of an example contact lens incorporating a tonometer system in accordance with the present disclosure.

FIG. 4B illustrates a top view of an example contact lens incorporating a tonometer system in accordance with the present disclosure.

FIG. 5A illustrates a cross-sectional view of an example contact lens including a test body in an initial position on an eye in accordance with the present disclosure.

FIG. 5B cross-sectional view of an example contact lens including a test body in an expanded position on an eye in accordance with the present disclosure.

FIG. 5C illustrates a cross-sectional view of an example contact lens including a test body transitioning from an expanded state to an initial state to measure the absolute intraocular pressure of an eye in accordance with the present disclosure.

FIG. 5D is a graph of a calibration curve of a contact lens on a porcine eye.

FIG. 6 is a block diagram of an example system for wirelessly determining the intraocular pressure of an eye in accordance with the present disclosure.

FIG. 7 is a block diagram of an example method of making a contact lens incorporating a variable capacitance sensor in accordance with the present disclosure.

FIG. 8 is a block diagram of an example method of making a contact lens incorporating a variable capacitance sensor in accordance with the present disclosure.

FIG. 9 illustrates a block diagram of an example method of manufacturing a test body on a contact lens in accordance with the present disclosure.

FIGS. 10A-10F illustrate schematic diagrams of the steps of an example method of manufacturing a tonometer system including a test body on a contact lens in accordance with the present disclosure.

FIGS. 11A-11H illustrate schematic diagrams of the steps of an example method of manufacturing a tonometer system including a test body incorporated into a contact lens in accordance with the present disclosure.

Throughout the drawings, identical reference numbers designate similar, but not necessarily identical, elements.

### DETAILED DESCRIPTION

The disclosures presented herein describe various apparatuses, systems, and methods for determining an intraocular pressure of an eye using a contact lens worn on the eye. As described in greater detail in the embodiments disclosed herein, these apparatuses, systems, and methods can use a contact lens and a sensor utilizing at least one of a capacitance measurement, a deflection measurement, or a magnetic field measurement. These measurements can be correlated to at least one of an absolute intraocular pressure of the eye or a relative intraocular pressure of the eye.

For example, in some embodiments, the principles described herein include incorporating a variable capacitance sensor into a contact lens that can be worn on a user's eye. While the contact lens is worn on the user's eye, a relative measurement of the intraocular pressure of the eye can be wirelessly measured and monitored by detecting changes in the mechanical strain of the contact lens via the variable capacitance sensor.

In some examples, the variable capacitance sensor can include one or more layers of transparent material and can be disposed on an outer surface of the contact lens. For example, the variable capacitance sensor can include a first conductive layer, a second conductive layer, and a dielectric layer disposed between the first and second conductive layers. One or more of the conductive layers can include a polymer, a metal, a microcomposite material, a nanocomposite material, any appropriate material, or combinations thereof. For example, the conductive layers can be transparent polymer layers and can include, for example, poly(3,4-ethylenedioxythiophene) polystyrene sulfonate. The dielectric layer can be a transparent polymer layer and can include, for example, polydimethylsiloxane. In some examples, one or more of these layers can be deposited on the contact lens by a number of deposition process, for example, cast molding, printing, or spin casting. In some examples, the layers can be deposited on a mold and can then be stamped onto the contact lens. In some examples, a diffusion or migration barrier is included between one or more layers to avoid material contamination between the layers. For example, a diffusion or migration barrier can be included between a conductive layer and dielectric layer to prevent the diffusion or migration of materials there between.

In some embodiments, the variable capacitance sensor including first and second conductive layers with a dielectric layer disposed there between can function as a capacitor. For example, the variable capacitance sensor can be a parallel plate capacitor, with the first and second conductive layers serving as parallel plates. In this example the capacitance of the variable capacitance sensor is related to the separation distance between the first and second conductive layers, or, in other words, the thickness of the dielectric layer disposed there between. In some examples, as the contact lens, and variable capacitance sensor disposed thereon, is bent or stretched due to, for example, the expansion or retraction of an eye from changes in intraocular pressure, the dielectric layer can experience a corresponding change in thickness. This change in thickness, for example due to stretching of the dielectric material, can thereby result in a change in the capacitance of the capacitor including the variable capacitance sensor.

The variable capacitance sensor can further include an antenna structure. In some examples, the antenna structure can be incorporated into the topmost conductive layer of the capacitor including the variable capacitance sensor. Thus, in some examples, the variable capacitance sensor can be an electrical oscillator formed by the capacitor, the antenna, which has a constant inductance, and the natural resistance of the variable capacitance sensor. In some examples, this electrical oscillator can have a natural frequency which is dependent on, or corresponds to the mechanical strain of the variable capacitance sensor. Thus, in some examples, where the variable capacitance sensor is disposed on the contact lens, the mechanical strain of the contact lens, or how much the contact has been stretched by the eye, can be measured by detecting the natural frequency of the electrical oscillator including the variable capacitance sensor. In some examples, the contact lens and/or variable capacitance sensor can further include a temperature sensor. In some embodiments, data from the temperature sensor can be used to mathematically compensate for the natural thermal expansion of materials in the contact lens in order to obtain a more accurate reading.

In some examples, the natural frequency of the variable capacitance sensor can be wirelessly detected by an electronic device. For example, an electronic device can send a signal having a signal frequency to the contact lens such that the contact lens sends a response signal when the signal frequency matches the natural frequency of the electrical oscillator including the variable capacitance sensor. In this way, the mechanical strain of the contact lens, and thus relative intraocular pressure of the eye, can be wirelessly measured. Further, the contact lens may not include a battery or integrated circuit, thereby simplifying manufacturing and reducing costs. In some examples, the electronic device can have a wireless remote powering system and can use a far field electromagnetic coupling method to transmit power to the contact lens. In some examples, the electronic device can have a wireless remote powering system and can use an inductive coupling, or near field, method to transmit power to the contact lens. In some examples, communication between the contact lens and electronic device can occur via a half duplex or full duplex scheme. That is, in some examples, both power and data can be wirelessly transmitted between the contact lens and an electronic device via a single wireless connection. However, in some examples, power can be transmitted by one method or connection and data can be transmitted by a second method or connection.

Additional principles described herein include incorporating a tonometer system including a test body into a contact lens that can be worn on a user's eye and utilizes a deflection measurement to determine at least one of an absolute or a relative intraocular eye pressure. In some embodiments, while the contact lens is worn on the user's eye a tonometer system can wirelessly measure the absolute intraocular pressure of the eye by measuring the rebound of the test body on the eye surface. In some examples, the rate of deceleration of the test body caused by the physical properties of the eye is detected and used to calculate the absolute intraocular pressure of the eye. In some examples, the rate of return of the eye and/or lens from an expanded state to a non-expanded state is measured and used to calculate the absolute intraocular pressure of the eye. The rate of deceleration of the test body and/or the rate of return of the contact lens or eye can be measured by a sensor which is situated on or incorporated into the contact lens and which can wirelessly transmit the relevant data to a secondary device, such as an electronic device. In some examples, the tonometer system can include the sensor. In some examples, this sensor can be a variable capacitance sensor, and can measure the mechanical strain of the contact lens while on the eye.

In some examples, the contact lens can comprise lens material and a test body in contact with the lens material. The test body can be disposed on a surface of the lens material, or it can be incorporated into the lens material. In some embodiments, the test body can be positioned outside of the optic zone of the contact lens that is configured to correct the user's vision. In this type of example, the test body may not interfere with or obstruct a user's vision while wearing the contact lens. In some examples, a tonometer system can include a test body to exert force on the eye. In some examples, the test body can be a selectively expandable material which can exert a force on the eye when the expandable material is in an expanded state. That is, the expandable material can expand under certain conditions and can return to an initial state when those conditions are stopped or removed. For example, in some examples, the selectively expandable material can be a magnetoresponsive elastomer. The magnetoresponsive elastomer material can expand in the presence of a magnetic field, and can return to an initial state when the magnetic field is removed.

In certain embodiments, the contact lens can further include a sensor, for example as a component of the tonometer system that can detect certain characteristics of the test body and/or contact lens to determine the absolute intraocular pressure of the eye. For example, the contact lens can include a sensor that can measure the amount of time it takes for the test body to return from an expanded state to the initial state. In some examples, the contact lens can include a sensor that can measure the deceleration of the test body caused by the eye as it enters the expanded state. The sensor can wirelessly communicate this information to a secondary device, such as an electronic device, which can manage sensor data in real time and calculate the absolute intraocular pressure of the eye, a relatively pressure of the eye, another parameter of the eye, or combinations thereof. In some examples, the sensor can be a transparent variable capacitance sensor that is capable of measuring the mechanical strain of the contact lens on the eye by correlating its changes in capacitance when the expandable material is expanded verses when the expandable material is in the initial state. As used herein, the term “absolute intraocular pressure” can refer to the total fluid pressure inside an eye, whereas the term “relative intraocular pressure” can refer to an amount of deviation or fluctuation from a baseline intraocular pressure value.

A contact lens including a test body can be manufactured by a variety of methods, including spin casting, dip coating, printing, stamping, or some combination thereof. In certain embodiments contact lens material can be provided and the test body can be deposited or formed on the lens material in a layer-by-layer deposition process. Thus, the test body can comprise one or more polymer layers which are deposited or formed on the lens material by printing, spin casting, and/or dip coating. However, in some other embodiments the contact lens can be manufactured by a stamping process, and the test body is deposited or formed on a mold, attached to a stamp or tool, and then deposited on the contact lens material. The stamping process can greatly reduce the processing or manufacturing time for the contact lens because the test body can be deposited or formed separately from the lens material, allowing for parallel processing lines. Once both the lens material and test body have been formed, they can be joined by stamping. In some examples, the contact lens body is formed through a spin casting process, and at least a portion of the circuitry for measuring the eye's intraocular pressure is manufactured separately and joined to the spin casted contact lens body.

In still other examples, additional principles described herein include incorporating a micromagnet or a micromagnet array as a test body into a contact lens that can be worn on a user's eye. These or other examples use a magnetic field measurement of the micromagnet or the micromagnet array to determine at least one of an absolute or a relative intraocular eye pressure. Similar to other embodiments, the contact lens can comprise lens material and a micromagnet test body in contact with the lens material. The micromagnet test body can be disposed on a surface of the lens material, or it can be incorporated into the lens material. In some embodiments, the micromagnet test body can be positioned outside of the optic zone of the contact lens that is configured to correct the user's vision. In this type of example, the micromagnet test body may not interfere with or obstruct a user's vision while wearing the contact lens. A gradiometer can be used to measure a measurement of the magnetic field around micromagnet test body, and correlate that measurement to at least one of an absolute or a relative intraocular eye pressure. For example the oscillation of the test body can be measured and correlated to an absolute or a relative intraocular eye pressure

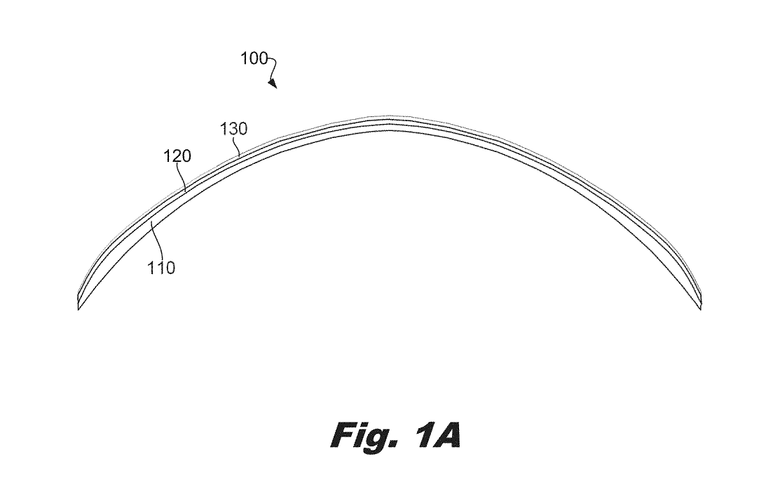

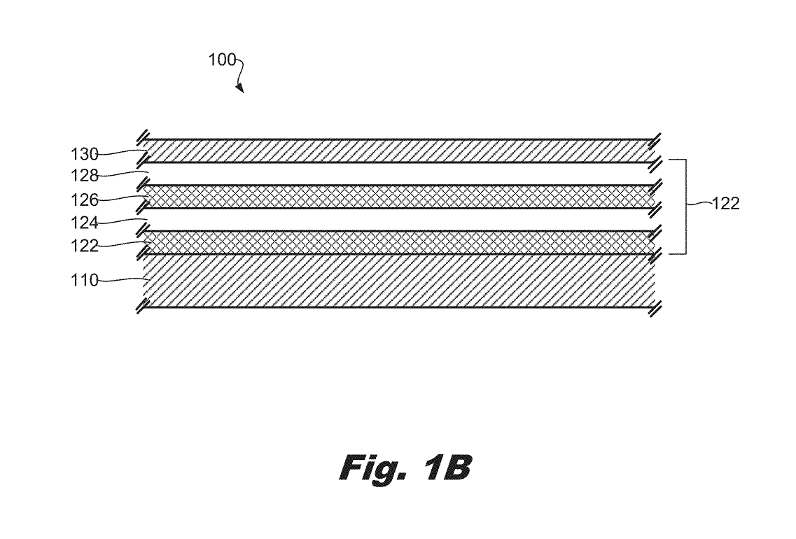

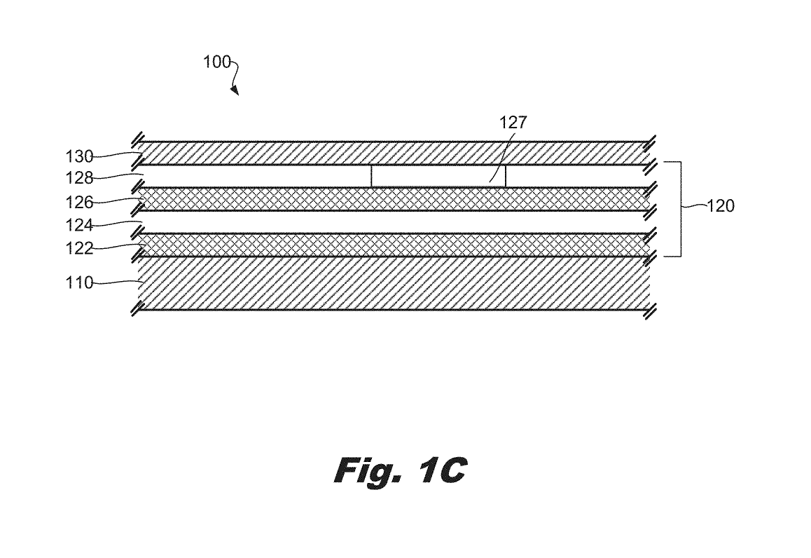

FIG. 1A depicts an example of a contact lens 100 including a lens material 110 and a sensor 120 disposed thereon. In some embodiments, the sensor can include a variable capacitance sensor 120. The lens material 110 can include any material suitable for use as a contact lens as is now know or can be developed in the future. That is, in some examples, the lens material 110 can include a typical hydrogel contact lens as is known in the art. For example, in some embodiments the lens material 110 can include a transparent polymer material, such as a hydrogel. In some examples, the lens material 110 can include a silicone hydrogel material. The contact lens material 110 can include an optic area or zone positioned at the center of the contact lens 100. The optic zone is typically about the same size as the pupil of the eye in low-light conditions, for example the optic zone can have a diameter of about 10 millimeters. The optic zone contains the corrective power of the contact lens 100, if any corrective power is present.

Further, the contact lens 100 can include an encapsulation layer 130 disposed over the variable capacitance sensor 120. The variable capacitance sensor 120 and the encapsulation layer 130 can have a combined thickness of between approximately 20 micrometers and approximately 50 micrometers, such as between approximately 20 micrometers and approximately 30 micrometers, between approximately 30 micrometers and approximately 40 micrometers, between approximately 40 micrometers and approximately 50 micrometers, less than approximately 50 micrometers, less than approximately 45 micrometers, less than approximately 40 micrometers, less than approximately 35 micrometers, less than approximately 30 micrometers, or less than less than approximately 25 micrometers.

In some embodiments and as shown in FIG. 1B, the variable capacitance sensor 120 can contain at least a layer of conductive material 124, also referred to as conductive layer 124, and a layer of dielectric material 126, also referred to as dielectric layer 126, overlying the layer of conductive material 124. In some examples, the layer of dielectric material 126 can be disposed directly on the layer of conductive material 126. In some examples, the variable capacitance sensor 120 can include a first conductive layer 124, a dielectric layer 126 overlying the first conductive layer 124, and a second conductive layer 128 overlying the dielectric layer 126. The second conductive layer 128 can be disposed directly on the dielectric layer 126.

The conductive layer or layers 124, 128 and the dielectric layer 126 can cover or be disposed over a substantially similar area of the underlying lens material 110. That is, the conductive layer or layers 124, 128 and the dielectric layer 126 can have a substantially identical shape and/or border when viewed from above. In some examples, the conductive layer or layers 124, 128 and the dielectric layer 126 can have an approximately circular, elliptical, or ovular shape on the lens material 110. However, in some other examples, the conductive layer or layers 124, 128 and the dielectric layer 126 can include any shape and/or boundary as is suitable for use in the variable capacitance sensor 120 as described herein, for example a conductive layer 124, 128 can have a half-moon shape. In some examples, a single conductive layers, for example conductive layer 124 or 128 can include two half-moon shapes separated from one another. The conductive and/or dielectric layers can be substantially continuous layers. In some examples, the conductive and/or dielectric layers may not be substantially continuous and can include one or more separate areas of the same layer.

In some examples, the conductive material including the layer or layers of conductive material 124, 128, can be a transparent polymer material. The conductive material can include a polymer mixture of two or more ionomers. In some examples, the conductive material can include a polymer or polymer mixture having aromatic cycles and/or double bonds. In some examples, the conductive material can include a polymer or polymer mixture including nitrogen and/or sulfur. In some examples, the conductive material can include a macromolecular salt. For example, in some embodiments the conductive material can include poly(3,4-ethylenedioxythiophene) polystyrene sulfonate (PEDOT:PSS). In some examples, the conductive material can include one or more additives, such as polyethylene glycol (PEG), for example to control or adjust the viscosity of the conductive material during processing.

In some other embodiments the conductive material can include a gel, such a hydrogel mixed with a suitable salt. For example, the conductive material can include a silicone hydrogel mixed with a salt to thereby form an ionic conductor. In some examples, the salt can be sodium chloride (NaCl). In some examples, where the conductive material include a hydrogel mixed with a salt the layer or layers forming the conductive material can advantageously have substantially the same or similar mechanical properties as the underlying lens material 110.

In some examples, the dielectric material including the dielectric layer 126 can include a transparent polymer material. The dielectric material can be an elastomer. In some examples, the dielectric material can include any transparent elastomer having a lower electrical and/or ionic conductivity than the conductive material. For example, in some examples, the dielectric material can include polydimethylsiloxane (PDMS).

As used herein, the term ‘conductive’ refers to the ability of the layer or material to act as an electrical and/or ionic conductor while the term ‘dielectric’ refers to the ability of the layer or material to act as an electrical or ionic insulator. When used herein in conjunction with one another, the terms ‘conductive’ or ‘conducting’ refers to the fact that the ‘conductive’ material has a higher electrical and/or ionic conductivity than the ‘dielectric’ material.

In some embodiments the variable capacitance sensor 120 can further include an additional layer of dielectric material 122 disposed below the first conductive layer 124. Thus, in some embodiments the first conductive layer 124 of the variable capacitance sensor 120 can be disposed directly on the lens material 110, for example on the outer surface of the lens material 110, however in some other embodiments the variable capacitance sensor 120 can include a lower dielectric layer 122 that is disposed directly on the lens material 110. In some examples, this lower dielectric layer 122 can function as a substrate layer during manufacturing of the variable capacitance sensor 120 as further described herein.

In some examples, where the variable capacitance sensor 120 can include two or more conductive layers 124, 128, each conductive layer can include the same conductive material, or each layer can include a different conductive material from any other conductive layer. Similarly, in examples where the variable capacitance sensor 120 can include two or more dielectric layers 122, 126, each dielectric layer can include the same dielectric material, or each layer can include a different dielectric material from any other dielectric layer.

In some examples, the contact lens 100 can further include an encapsulation layer 130 disposed over the variable capacitance sensor 120. The encapsulation layer 130 can be in direct contact with the variable capacitance sensor 120. In some examples, the encapsulation layer 130 can include a polymer material, such as a hydrogel. In some examples, the lens material 110 can include a silicone hydrogel material and can be the same material as the lens material 110. The encapsulation layer 130 can have a thickness of from about 0.1 micrometers to about 20 micrometers, from about 0.5 micrometers to about 15 micrometers, or from about 1 micrometers to about 10 micrometers.

The variable capacitance sensor 120 can have a thickness on the lens material 110 of from about 10 micrometers to about 100 micrometers, or from about 20 micrometers to about 50 micrometers. It has been advantageously found that when the variable capacitance sensor 120 has a thickness of less than about 100 micrometers, specifically less than about 50 micrometers, the variable capacitance sensor 120 is able to function well in detecting the relative intraocular pressure of an eye, while the contact lens 100 including the variable capacitance sensor 120 perform in a substantially identical way with respect to user comfort and visions correction to a typical contact lens that does not include a sensor. In some examples, the one or more conductive layers which include the variable capacitance sensor 120 can each have a thickness of from about 0.1 micrometers to about 20 micrometers, from about 0.5 micrometers to about 15 micrometers, or from about 1 micrometer to about 10 micrometers. Similarly, the one or more dielectric layers 122, 126 which can include the variable capacitance sensor 120, can have a thickness of from about 0.1 micrometers to about 20 micrometers, from about 0.5 micrometers to about 15 micrometers, or from about 1 micrometer to about 10 micrometers.

Turning to FIG. 1C, in some embodiments, the contact lens 100 can also include a micromagnet 127. The micromagnet 127 can be positioned between the lens material and the encapsulation layer 130. According to some embodiments, the micromagnet 127 described herein are positioned on the contact lens material 110 outside of the optic zone. In some examples, the micromagnet 127 can be positioned substantially adjacent to the optic zone, however in some other examples the micromagnet 127 can be positioned near an edge of the lens material 110, or any position therebetween.

In an example, the micromagnet 127 is positioned between the encapsulation layer 130 and the one or more dielectric layers 122, 126. For example, the micromagnet 127 can be positioned between the dielectric layer 126 and the encapsulation layer 130. In another example, the micromagnet 127 can be positioned between the conductive layer 128 and the encapsulation layer 130. In some embodiments, the micromagnet 127 can be positioned on or embedded at least partially within at least one of the one or more dielectric layers 122, 126 or the one or more conductive layers 128, 124. For example, the micromagnet 127 can be positioned on or embedded at least partially within the conductive layer 128 or the dielectric layer 126.

The micromagnet 127 is actuated by a magnetic actuator positioned outside the eye. The magnetic actuator is configured to bias or otherwise push the micromagnet 127 towards the eye to perform at least one of applanation or indentation of the eye. The micromagnet 127 can include a rare earth magnet, such as a sintered samarium cobalt magnet or neodymium iron boron. In some embodiments, the micromagnet 127 can include a coating to modify reflectance and enable time-of-flight proximity sensing. In some embodiments, the micromagnet 127 can include a single magnet, such as a ferromagnetic disc. A single micromagnet disc can be molded into the contact lens 100 and magnetized to a desired direction after polymerization of the contact lens 100. Alternatively, a single micromagnet disc can be magnetized prior to the polymerization of the contact lens using an additional previously magnetized disc.

In some embodiments, the micromagnet 127 can include a micromagnet array. The micromagnet array can be transparent, while the rigidity and the flexibility of the micromagnet are variable, depending upon the particle granulometry and array density. The micromagnet 127 can also include multiple layers of micromagnet arrays that are fabricated to transfer wireless power from the magnetic source to the contact lens 100. The micromagnet array can be integrated into the contact lens 100 during molding of the contact lens 100.

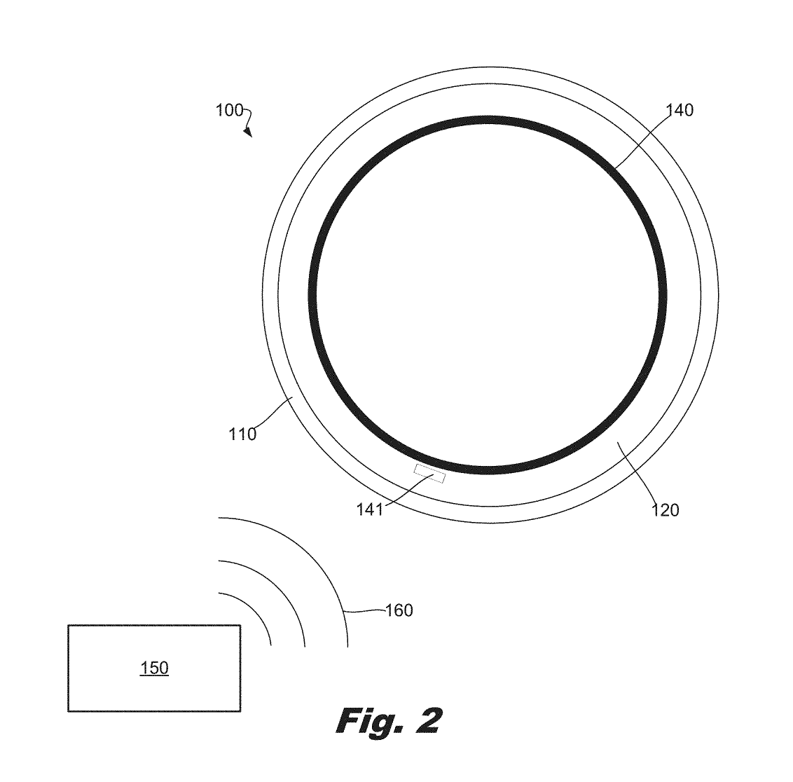

During applanation or indentation, as the magnetic actuator pushes the micromagnet 127 towards the eye, the layers of the variable capacitance sensor 120 will be mechanically compressed, thus changing the distance between the dielectric layers 122, 126 and the capacitance of the contact lens 100. As illustrated in FIG. 2, in some examples, the variable capacitance sensor 120 can also include an application specific integrated circuit (ASIC) 141. The ASIC 141 is configured to measure the capacitance in the variable capacitance sensor 120 before, during, and after applanation or indentation is performed using the magnetic actuator and the micromagnet 127.

As described herein, and as illustrated in FIG. 2, in some examples, the variable capacitance sensor 120 can include an antenna structure 140. The antenna structure 140 can include a loop or coil structure, as is well known in the art. However, other antenna designs are expressly contemplated herein. For example, any antenna design capable of functioning as described herein, and which can be incorporated into the variable capacitance sensor 120 as described herein, can be utilized as will be understood by the skilled artisan. In some examples, the antenna structure 140 can include conductive lines which include, for example, the coil or loop structure as shown. The conductive lines can include the conductive material used to form the conductive layers 124, 128 described herein and can have a line width of from about 25 micrometers to about 200 micrometers, or from about 50 micrometers to about 100 micrometers. Where the variable capacitance sensor 120 includes a capacitor, the antenna structure 140 can be electrically connected to each side of the capacitor to thereby form an electrical circuit. For example, where the variable capacitance sensor 120 includes a parallel plate capacitor including two conductive layers 124, 128, the antenna structure can be electrically connected to each of the conductive layers 124, 128 to thereby form an electrical circuit.

In some examples, the antenna structure 140 can be formed on the upper conductive layer 128, for example, by printing conductive material in the form of the antenna structure 140. In some examples, the antenna structure 140 can be formed by inkjet printing conductive material on the upper conductive layer 128. The antenna structure 140 can also be formed by a stencil process wherein conductive material is painted or applied into a stencil including the desired antenna structure 140, which is disposed over the conductive layer 128. Other methods of forming the antenna structure 140 can be utilized, as are known in the art or can be developed in the future. The antenna structure can have a thickness of from about 0.1 micrometers to about 20 micrometers, from about 0.5 micrometers to about 15 micrometers, or from about 1 micrometers to about 10 micrometers. In some examples, the antenna structure 140 can thus be incorporated into, or become a part of, the upper conductive layer 128 after it has been deposited or formed. However, in some other examples, an additional layer including dielectric material (not shown) can be deposited or formed over the upper conductive layer 128 and the antenna structure 140 can be formed on this additional dielectric layer, for example by printing or a stencil process.

As described herein, in some examples, the variable capacitance sensor 120 can include a capacitor having two parallel conductive layers 124, 128 with a dielectric layer 126, disposed there between. In these examples where the conductive layers 124, 128 act as the plates in a parallel plate capacitor, the capacitance (C) of the capacitor can be given by the equation:

$$C = \frac{{e}_{0} {e}_{r} A}{d} \qquad Equation 1$$

Where e₀ is the permittivity of free space, a constant, eᵣ is the relative permittivity of the dielectric layer 126, A is the effective surface area of the plates of the capacitor, that is, conductive layers 124, 128, and d is the thickness of the dielectric layer 126. When the contact lens 100 is subjected to a mechanical strain, for example during relative changes in intraocular pressure while the lens 100 is on the eye, the lens 100 will expand or contract with the eye. This expansion or contraction will cause changes in the area of the conductive layers 124, 128 (A) and in the thickness of the dielectric layer 126(d), and thus will cause corresponding changes in the capacitance (C). For example, an increase in intraocular pressure will cause the eye and lens 100 to expand, thereby causing an increase in the area of the conductive layers 124, 128 (A) and a decrease in the thickness of the dielectric layer 126(d). Similarly, a decrease in intraocular pressure will cause the eye and lens 100 to contract, thereby causing a decrease in the area of the conductive layers 124, 128 (A) and an increase in the thickness of the dielectric layer 126(d). The corresponding changes to the capacitance (C) can ultimately be detected as described herein, in order to determine the relative intraocular pressure of the eye.

However, rather than continuously measuring the area of thickness of the layers forming the capacitor, it was found that when a uniaxial force stretches the capacitor with a factor (λ), as occurs during changes in intraocular pressure of the eye, the capacitance (C) scales as:

C=C₀λ⁴ Equation 2

where C₀ is the original capacitance of the capacitor in an initial state. For example, the initial state can be an un-stretched state, where the lens does not experience tensile forces. In some examples, the initial state can be such that some tensile forces are exerted across at least a portion of the lens, for example when the lens is on an eye. By knowing the original capacitance (C₀) and measuring the capacitance (C) this scaling factor (λ) can be determined and, for example, transmitted to an external reader device in order to determine the relative intraocular pressure of the eye. This is possible because the scaling factor (λ) is proportional to the mechanical strain on the contact lens 100, which is proportional to the intraocular pressure of the eye. In some examples, the scaling factor (λ) can be linearly related to the mechanical strain on the contact lens 100, however in other examples, the scaling factor (λ) can have a non-linear relationship with the mechanical strain on the contact lens.

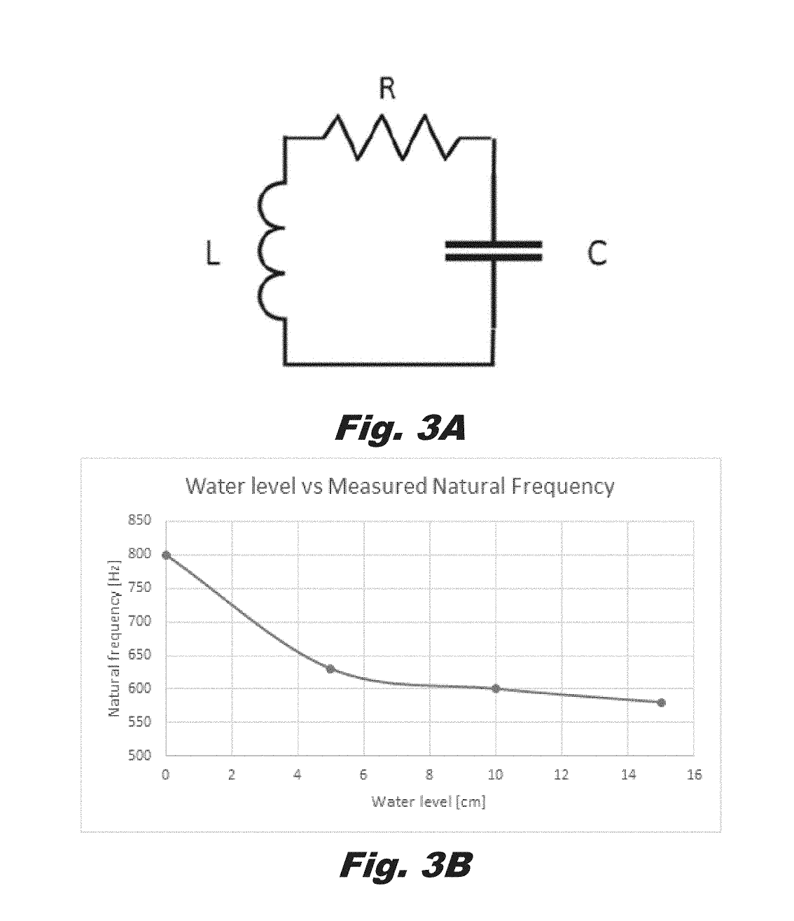

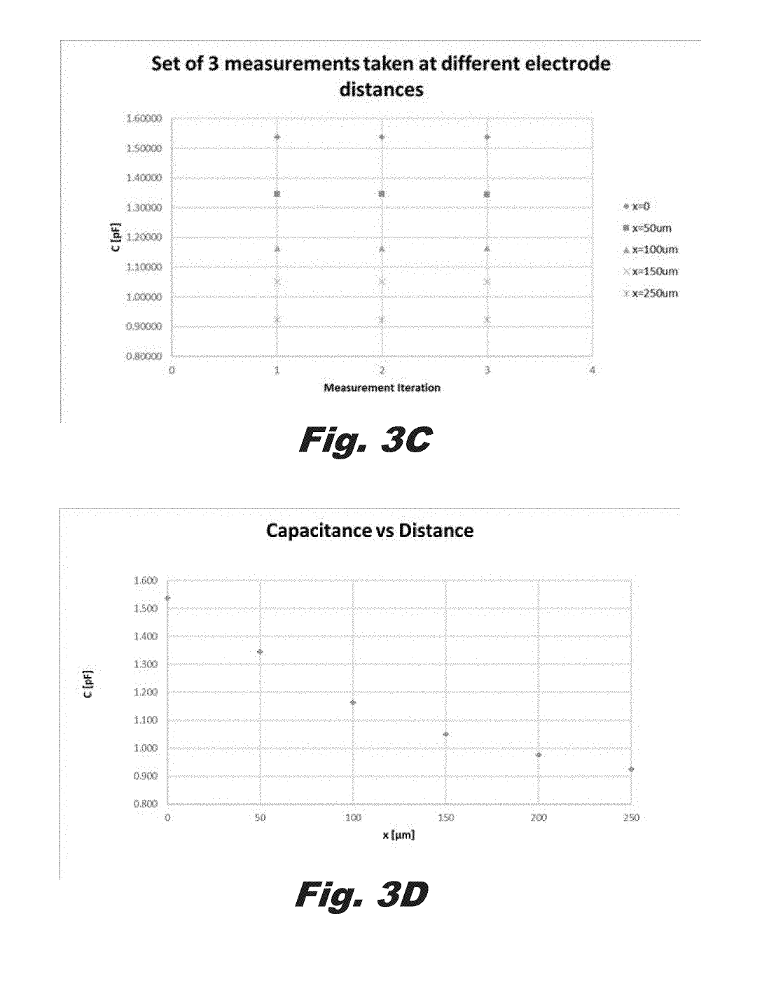

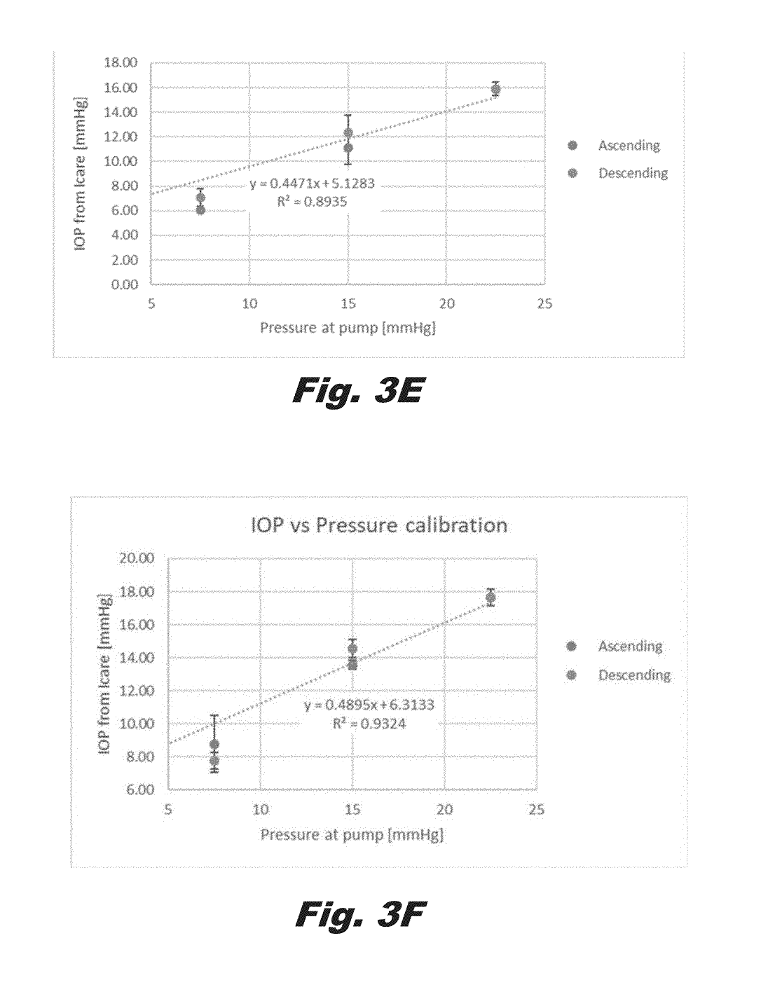

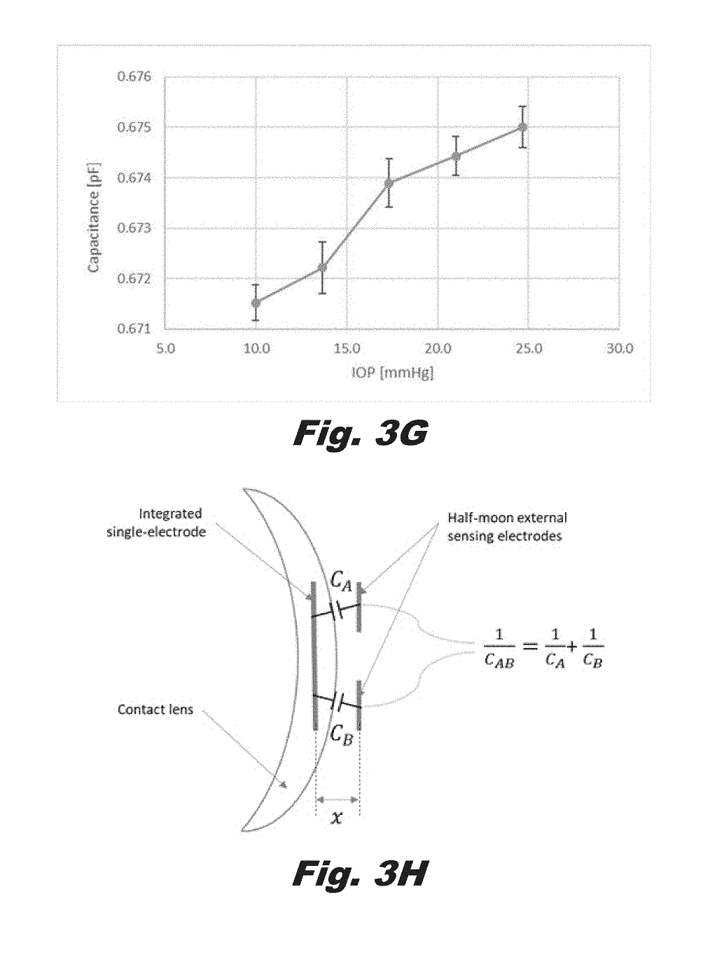

In examples where the variable capacitance sensor 120 includes an antenna structure 140 electrically connected to the two conductive layers 124, 128 including the capacitor, an electrical oscillator can be formed. The electrical oscillator can be an LCR oscillator which can be conceptualized as an inductor, a capacitor, and a resistor connected in series. Here, the inductance (L) is constant and given by the structure of the antenna 140, the capacitance (C) is described by Equation 1 and Equation 2, and the resistance (R) is determined by the conductivity of the conductive layers 124, 128. FIG. 3A illustrates an example circuit diagram of the LCR oscillator including variable capacitance sensor 120 and antenna structure 140. The natural frequency (f) of this oscillator is given by the equation:

$$f = \frac{1}{2 π \sqrt{LC}} \qquad Equation 3$$

However utilizing Equation 2 to describe the capacitance (C) allows for the natural frequency (f) of oscillator to be written as:

$$f = \frac{1}{2 π {λ}^{2} \sqrt{{LC}_{0}}} \qquad Equation 4$$

Accordingly, the scaling factor (λ), and thus relative intraocular pressure of the eye, can be determined by measuring or detecting the natural frequency (f) of the electrical oscillator formed from the capacitor including the variable capacitance sensor 120 and antenna structure 140.

Referring again to FIG. 2, the natural frequency of such an electrical oscillator can be measured using the ASIC 141 on the contact lens 100. For example, as described above, the ASIC 141 can measure the capacitance during before, during, and/or after the magnetic actuator and micromagnet 127 perform applanation or indentation on the eye. Using the measured capacitance, the ASIC 141 can then determine natural frequency. Alternatively, the capacitance measured by the ASIC 141 can be transmitted via the antenna 140 and a wireless signal 160 to a separate electronic device 150. The electronic device 150 or some other electronic device can then determine the natural frequency using the capacitance measured by the ASIC 141.

Alternatively, in some embodiments, the natural frequency of such an electrical oscillator can be measured from a separate electronic device 150, such as a vector network analyzer (VNA). The electronic device 150 can send a wireless signal 160 to the contact lens 100, which upon receipt of the wireless signal 160 by the antenna structure 140 can send a response signal to the electronic device 150, as described further herein. The signal received by the electronic device 150 can contain information such as the natural frequency (f) of the electrical oscillator in the contact lens 100 which can then be used to determine the relative intraocular pressure of the eye. In some embodiments, utilizing a separate electronic reader device 150 to determine the natural frequency (f) of the electrical oscillator allows for a wireless measurement of the relative intraocular pressure of the eye, via the contact lens 100, without the need for a power source, such as a battery, or an integrated circuit such as an ASIC on the lens 100. Thus, in some examples, the lens 100 does not include a power source or an integrated circuit.

To test how natural frequency changed with application of pressure, small amounts of pressure were applied to a contact lens with graphite. Each water level of pressure was held for 1 minute, during which the natural frequency was measured. As demonstrated in FIG. 3B, it was observed that the natural frequency of the contact lens changed with varying amounts of pressure applied to the contact lens. Specifically, the measured natural frequency of the contact lens drops as the pressure applied to the contact lens increases. It is noted that the natural frequency was detected to be around 4.1395 GHz, and the y-axis of the graph shown in FIG. 3B present only the last three digits of the measurement. For example, with no pressure (a water level of 0 cm), the natural frequency was 4.139500800 GHz. At a water level pressure of 5 cm, the natural frequency dropped to between approximately 4.139500625 GHz and 4.139500640 GHz. At a water level pressure of 10 cm, the natural frequency dropped to approximately 4.139500600 GHz. At a water level pressure of 15 cm, the natural frequency of the contact lens dropped to between approximately 4.139500575 GHz and 4.139005590 GHz.

To demonstrate how capacitance between sensors in a contact lens can be used to determine intraocular eye pressure, a contact lens including an integrated single electrode was placed on a porcine eye in a laboratory. A half-moon configuration of a second internal electrode was mimicked using a first external half-moon sensing electrode and a second external half-moon sensing electrode, as illustrated in the cross-sectional view of FIG. 3H. A fluidic system including a pump was connected to the porcine eye to regulate intraocular eye pressures of the porcine eye, and the porcine eye was inserted into a support in the laboratory.

A first capacitance (CA) was measured between the first external half-moon sensing electrode and the integrated single electrode, and a second capacitance (C_{B}) was measured between the second external half-moon sensing electrode and the integrated single electrode. Overall capacitance could be determined using

$$\frac{1}{{C}_{AB}} = \frac{1}{{C}_{A}} + \frac{1}{{C}_{B}}$$

Changing the intraocular eye pressure of the porcine eye induced volume deformation of the globe of the porcine eye, which was reflected by a change in the distance x between the integrated single electrode and the two external half-moon sensing electrodes. It was observed that this change in distance also caused a change in total capacitance (C) value. Accordingly,

$${C}_{Total} \frac{1}{x}$$

Data was acquired when the two half-moon electrodes were placed less than 1 mm from a single electrode, which at first was not integrated in any contact lens. As shown in the graph of FIG. 3C, a set of three measurements was taken at different electrode distances to validate measurements of the test setup. The different electrode distances included 0 micrometers, 50 micrometers, 100 micrometers, 150 micrometers, and 250 micrometers. Capacitance remained substantially unchanged during each of the three iterations at each respective distance, as shown in FIG. 3C.

Total capacitance C also was measured as a function of distance x between the integrated single-electrode and the two external half-moon sensing electrodes, with the results shown in the graph of FIG. 3D. As demonstrated in the graph of FIG. 3D, capacitance decreased from between 1.500 and 1.600 pF at a distance of 0 micrometers to less than 1.000 pF at distances of 200 micrometers and 250 micrometers.

A reference sensor (ICARE tonometer) was used to calibrate actual intraocular eye pressure of the porcine eye with applied pressure from the pump of the fluidic system connected to the porcine eye. The ICARE tonometer measures deceleration and rebound time of a probe hitting the eye to calculate intraocular eye pressure of the eye. Calibration was performed on a first day (FIG. 3E) and on a second day (FIG. 3F) by determining intraocular eye pressure of the porcine eye with the ICARE tonometer at fluidic system pump pressures of 7.5 mmHg, 15 mmHg, and 22.5 mmHg.

Data from the two days of testing shown in FIGS. 3E and 3F was used to create a calibration curve for determining capacitance variation as a function of intraocular pressure of the eye, shown in FIG. 3G. Each point in FIG. 3G is an average of 200× raw capacitance values sampled at 4.6 Hz, and the error bars represent the standard deviation of these 200× values for each point. As demonstrated in FIG. 3G, then, measuring capacitance between two external electrodes and a third electrode in a contact lens can be useful in determining ocular hypertension and/or glaucoma, for example, in the eye of a wearer when an increase in the capacitance from (0.675 pF) to 0.675 pF, indicates an increase in the intraocular pressure of the eye from near 10 mmHg to near 25 mmHg.

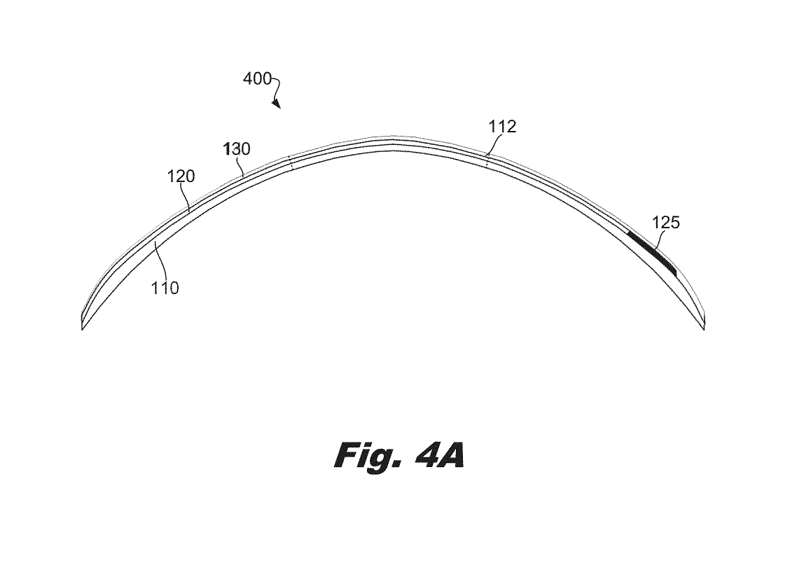

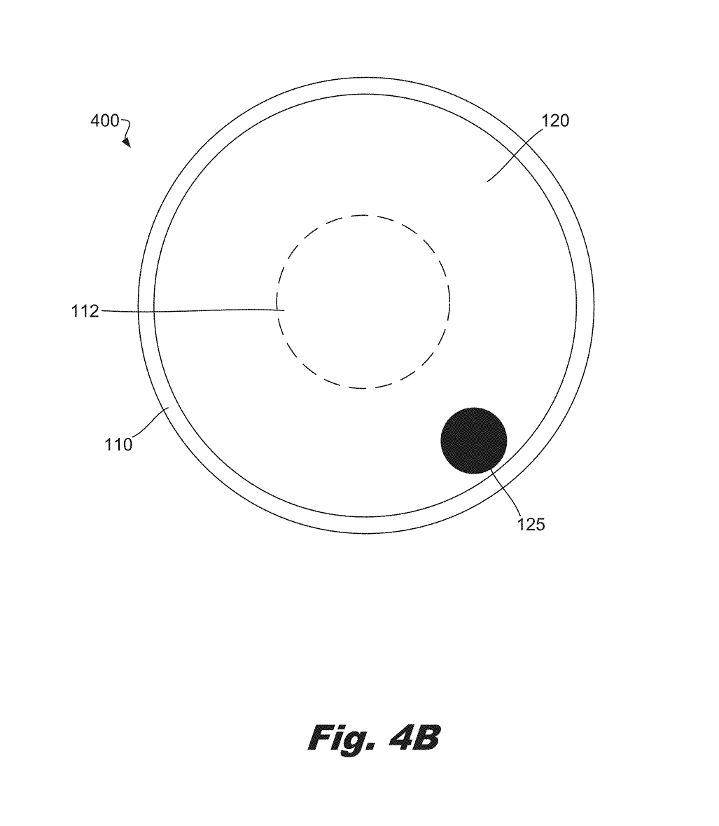

In many embodiments, a deflection measurement using a sensor in the contact lens can be used to determine at least one of an absolute or a relative intraocular eye pressure of an eye. FIGS. 4A and 4B depict an additional example of a contact lens 400 including a lens material 110, a sensor 120, and an encapsulation layer 130. The contact lens 400 can be utilized to determine an absolute intraocular pressure of the eye of a wearer. The contact lens 400 can also include a tonometer system including a test body 125 disposed thereon. The test body 125 can be disposed outside of the optic zone 112 of the contact lens. The lens material 110 can include any material suitable for use as a contact lens. That is, in some examples, the lens material 110 can include a typical hydrogel contact lens. For example, in some embodiments, the lens material 110 can include a transparent polymer material, such as a hydrogel. In some examples, the lens material 110 can include a silicone hydrogel material.

In some embodiments, the test body 125 can be disposed completely or partially within the optic zone 112 of the contact lens 400. In some of these examples, the test body 125 can be transparent and may not distort or interfere with the user's vision, at least when in an initial state. In some examples, however, a test body 125 positioned at least partially within the optic zone 112 of the contact lens 400 can interfere with or distort a user's vision when in an expanded state and/or the initial state.

The test body 125 can selectively exert the force on the eye sufficient to obtain an intraocular pressure value via rebound tonometry. In some embodiments, the test body 125 can be a selectively expandable material. That is, the test body 125 can be a material that expands under a predetermined condition or set of conditions, and returns to its initial state when the condition or conditions are removed. In certain embodiments, the expandable material can be a polymer material, and in some examples, the expandable material can be an elastomer. In some embodiments, the expandable material can be a hydrogel material.

In some examples, the expandable material can include a magnetoresponsive material that expands in the presence of a magnetic field and returns to the initial state when no longer in the presence of the magnetic field or within the presence of a magnetic field with a sufficient strength to expand the material. In these examples, the expandable magnetoresponsive material can include a polymer film including a plurality of molecular microchains which can be preferentially aligned with a magnetic field when the material is exposed to a magnetic field of sufficient strength. In some embodiments the expandable magnetoresponsive material can include aligned magnetic microchains throughout an entire thickness of the polymer film. In some embodiments, the aligned magnetic microchains can be formed upon the magnetophoretic transport and assembly of microparticles during polymer curing of the expandable magnetoresponsive material, for example, during formation of the test body 125.

In some embodiments, the expandable magnetoresponsive material can include a plurality of magnetic nanocrystals embedded in a polymer matrix, such as a polyvinyl alcohol (PVA) matrix. In some embodiments, the expandable magnetoresponsive material can include an elastomer, such as a silicon elastomer. In some examples, the elastomer can be a matrix in which magnetic microchains are dispersed. In some embodiments the magnetic microchains can be formed of particles having an average size of less than about 500 microns, less than about 250 microns, less than about 100 microns, less than about 50 microns, less than about 10 microns, or smaller. In some embodiments, the particles forming the magnetic microchains can be ferromagnetic particles, such as metallic ferromagnetic alloy particles. In some examples, these particles can include one or more of Nd, Fe, Pr, Co, B, Dy, Ga, or other elements. In some embodiments, the magnetic particles can be from about 1 wt % to about 50 wt % of the cured magnetoresponsive material. In some embodiments, magnetic microchains can be formed in an elastomeric matrix by applying an external magnetic field to the magnetoresponsive material while it is being cured or formed so that the magnetic particles are transported and aligned to form microchains having a substantially uniform orientation throughout the cured elastomeric matrix.

In some embodiments, when the expandable magnetoresponsive material is not exposed to a magnetic field of sufficient strength to expand the material, for example less than about 100 mT, less than about 10 mT, or less than about 1 mT or lower, the material can include a homogenous dispersion of microparticles or polymer blocks.

In some examples, the tonometer system and/or test body 125 do not include separate mechanical moveable parts. That is, in some embodiments the tonometer system and/or test body 125 may not include parts which move or slide with respect to one another. For example, in some examples, a tonometer system and/or test body 125 may not include a slidable or moveable central piece, such as a magnet and housing piece, such as a coil, through which the central piece can move. In some embodiments, the tonometer system and/or test body 125 may not include a permanent magnet and/or an electromagnet.

The test body 125 can have a diameter in an initial state of from about 1 millimeters to about 3 millimeters. In some examples, the test body 125 can have a diameter of about 2 millimeters. The test body 125 can have a thickness of about from about 25 micrometers to about 200 micrometers, or from about 50 micrometers to about 100 micrometers. It has been advantageously found that a test body 125 having a thickness of, for example, less than 100 micrometers allows for the ability to measure the absolute intraocular pressure of the eye without causing discomfort when the contact lens 400 is on the eye. Although the test body 120 is depicted in FIG. 4B as approximately circular in shape, other shapes are expressly contemplated. For example, in some embodiments the test body 125 can be elliptical, rectangular, or irregular shape. In some examples, the test body 125 can have a surface area of from about 1 square millimeter to about 10 square millimeters. In some examples, the test body 125 can have a surface area of about 4 square millimeters. In some situations, the surface area of the tonometer can be less than 10 percent of the contact lens' surface area, more than 10 percent of the contact lens' surface area, more than 20 percent of the contact lens' surface area, more than 30 percent of the contact lens' surface area, more than 50 percent of the contact lens' surface area, more than another percentage of the contact lens' surface area, or combinations thereof.

The contact lens material 110 can include an optic area or zone 112 positioned at the center of the contact lens 400. The optic zone 112 is typically about the same size as the pupil of the eye in low-light conditions, for example the optic zone can have a diameter of about 10 millimeters. The optic zone 112 contains the corrective power of the contact lens 400, if any corrective power is present. According to some embodiments, the test body 125 described herein is positioned on the contact lens material 110 outside of the optic zone 112. In some examples, the test body can be positioned substantially adjacent to the optic zone 112, however in some other examples the tonometer can be positioned near an edge of the lens material 110, or any position there between.

In some embodiments, the tonometer system of the contact lens 400 can further include a sensor 120. The sensor 120 can be in contact with the lens material 110. In some examples, the sensor 120 can detect and/or wirelessly transmit information regarding the rate of change of the mechanical strain of the contact lens 110 as the test body 125 transitions from an expanded state to an initial state. In some examples, the sensor 120 can detect and/or wirelessly transmit information regarding the deceleration of the test body 125 caused by the eye as the expandable material enters the expanded state. The sensor 120 can be, for example, a variable capacitance sensor. The sensor 120 can include a parallel plate capacitor and an antenna structure. The parallel plate capacitor of the sensor 120 can include at least a first transparent conductive layer and a second transparent conductive layer, with a dielectric layer disposed there between. The antenna connected to the sensor 120 can be electrically connected to each of the conductive layers to thereby form an electrical oscillator. In some examples, the natural frequency of the electrical oscillator including the sensor 120 can correspond to the amount of mechanical strain experienced by the contact lens 400, for example due to the expansion of the test body 125. Accordingly, a secondary electronic device, such as a vector network analyzer (VNA) can be used to detect the mechanical strain of the contact lens 400 via the sensor 120 in order to determine the rate of change of the mechanical strain and the absolute intraocular pressure of the eye.

In some examples, the contact lens 400 can further include an encapsulation layer 130 disposed over the test body 125 and/or sensor 120. The encapsulation layer 130 can be in direct contact with the test body 125. In some examples, the encapsulation layer 130 can include a polymer material, such as a hydrogel. In some examples, the encapsulation layer 130 can include a silicone hydrogel material and can be the same material as the lens material 110. The encapsulation layer 130 can have a thickness of from about 0.1 micrometers to about 20 micrometers, from about 0.5 micrometers to about 15 micrometers, or from about 1 micrometers to about 10 micrometers.

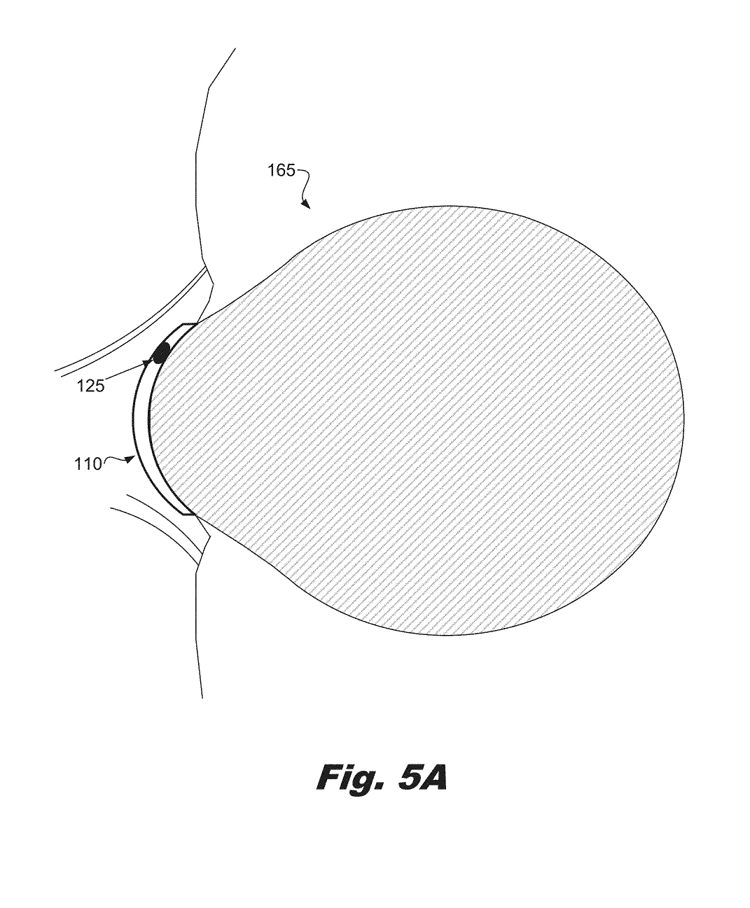

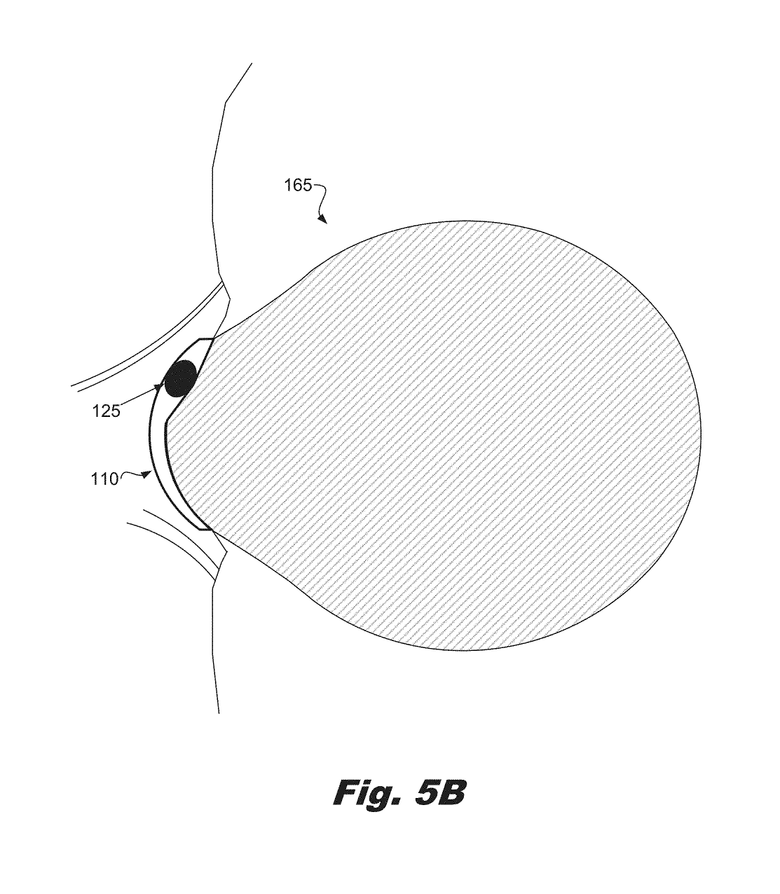

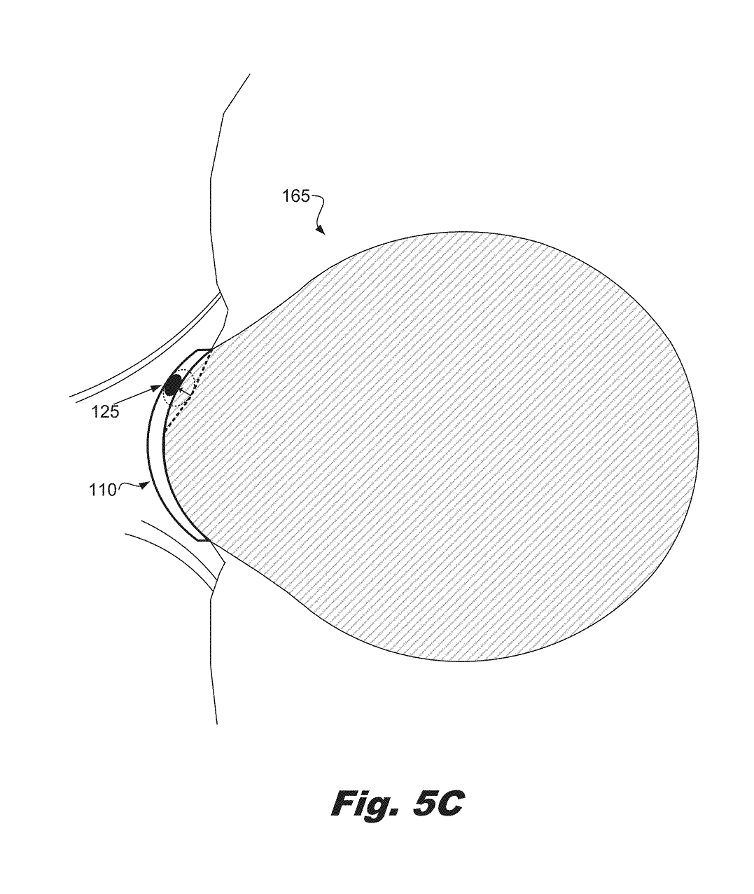

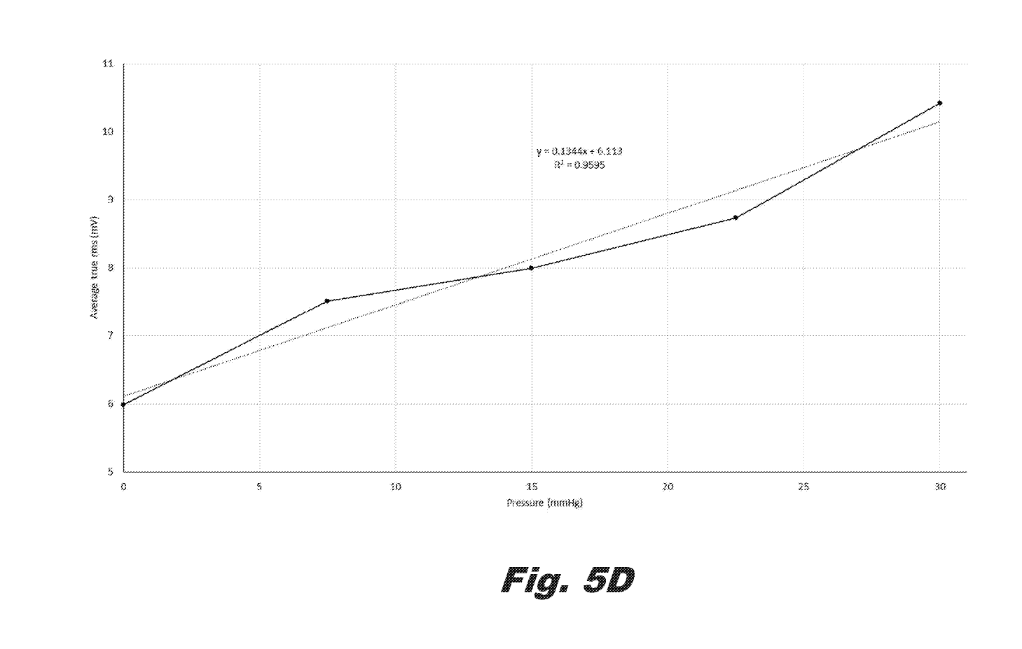

FIG. 5A shows a cross-sectional view of contact lens 400, including test body 125 in an initial state, positioned on the eye 165 of a user. As described herein, the test body 125 is positioned outside the optic zone 112 of the eye. FIG. 5B shows the contact lens 400 and test body 125 positioned on the eye 165 while the test body 125 is, for example, exposed to a magnetic field sufficient to expand the expandable magnetoresponsive material which can make up the test body. As can be seen in FIG. 5B, and as described herein, when the test body 125 is in an expanded state, it can exert a pressure on the eye 165 due to the expansion of the test body. This expansion and associated force can cause a slight deformation in the eye 165.

When the test body 125 is removed from, for example, a magnetic field and it can return to an initial state as the eye 165 rebounds to its original shape, as illustrated in FIG. 5C. As described herein, the amount of time involved for the test body 125 to transition back to the initial state, as shown in FIG. 5C, is measured by wirelessly detecting the change or rate of change in the mechanical strain experienced by the contact lens, which can then be used to determine the absolute intraocular pressure of the eye. It should be noted that the degree of expansion of the expandable material forming the test body 125, as depicted in FIGS. 5A-5C, can be exaggerated in order to better aid in the understanding of the present disclosure.

In many embodiments, a magnetic field measurement using a sensor in the contact lens can be used to determine at least one of an absolute or a relative intraocular eye pressure of an eye. For example, in some embodiments, the test body 125 includes a micromagnet. The micromagnet of the test body 125 can include a rare earth magnet, such as a sintered samarium cobalt magnet or neodymium iron boron. In some embodiments, the micromagnet of the test body can include a single magnet, such as a ferromagnetic disc. A single micromagnet disc can be molded into the contact lens 400 and magnetized to a desired direction after polymerization of the contact lens 400. Alternatively, a single micromagnet disc can be magnetized prior to the polymerization of the contact lens using an additional previously magnetized disc.

In some embodiments, the micromagnet of the test body 125 can include a micromagnet array. The micromagnet array can be transparent, while the rigidity and the flexibility of the micromagnet are variable depending upon the particle granulometry and array density. The micromagnet of the test body 125 can also include multiple layers of micromagnet arrays that are fabricated to transfer wireless power from the magnetic source to the contact lens 400. The micromagnet array can be integrated into the contact lens 400 during molding of the contact lens 400.

In some embodiments, the test body 125 includes a contrast agent configured to enable time-of-flight proximity sensing. The contrast agent can include at least one of a coating of a radiation source layer, a dye, or a tag. Alternatively, a surface of the test body 125, such as the micromagnet of the test body 125, can optionally include a coating to modify reflectance and enable time-of-flight proximity. The surface of the test body 125 can also include a selective specific transmitter time-of-flight system for position measurement. In these and other embodiments, the radiation source layer can be replaced by a radiation reflector material.

In some embodiments, an actuator outside the contact lens can be configured to bias or otherwise push the micromagnet of the test body 125 towards the eye to perform at least one of applanation or indentation of the eye. For example, when the micromagnet of the test body 125 is exposed to a magnetic field, the micromagnet of the test body 125 pushes towards the eye 165. Similar to the expandable magnetoresponsive material in FIG. 5B, the micromagnet of the test body 125 can exert a pressure on the eye 165. This force applied to the eye 165 responsive to movement of the micromagnet of the test body 125 can cause a slight deformation in the eye 165.

In some embodiments, the sensor 120 can measure the magnetic field of the test body 125, using a magnetometer or a gradiometer sensor, resulting in a voltage that can be correlated to a pressure of the eye 165. According to some examples, a contact lens including a micromagnet in the test body 125 may not include a sensor 120 in the contact lens, and instead use an external sensor in conjunction with the micromagnet in the test body 125. For example, a gradiometer can determine the magnetic field gradient of the test body 125, and determine a pressure on the eye 165 correlated with the displacement of the micromagnet integrated in the contact lens. FIG. 5D provides a graph demonstrating a calibration curve of a contact lens on a porcine eye. The gradiometer measured an average true root mean square (rms) of 5.988 mV with 0 Mbar (0 mmHg) of pressure, an average rms of 7.513 mV with 10 Mbar (7.50062 mmHg) of pressure, an average rms of 7.995 mV with 20 Mbar (15.00124 mmHg) of pressure, an average rms of 8.729 mV with 30 Mbar (22.50186 mmHg) of pressure, and an average rms of 10.42 mV with 40 Mbar (30.00248 mmHg) of pressure.

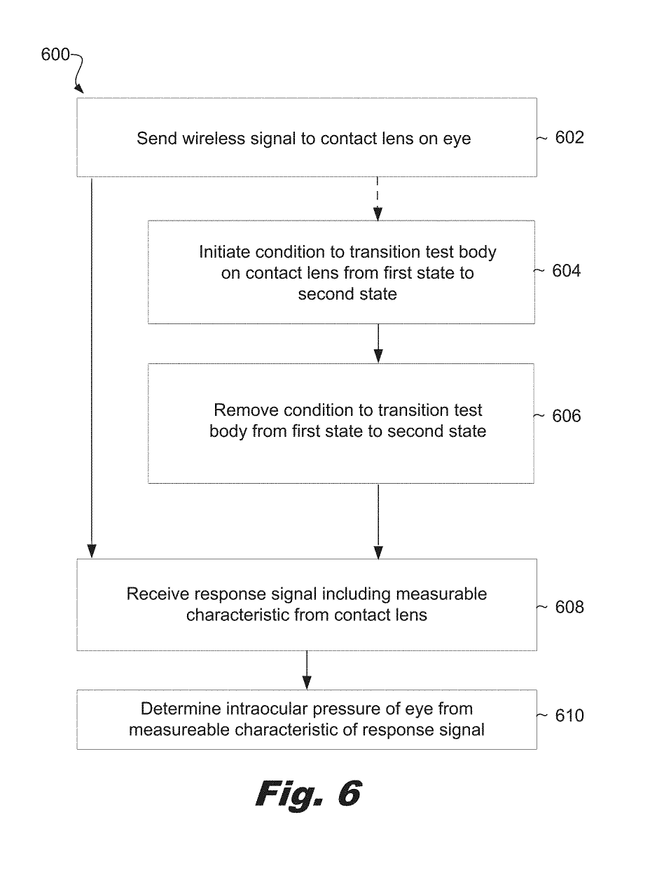

FIG. 6 illustrates an example of a method 600 of wirelessly determining the intraocular pressure of an eye. In this example, the method 600 includes sending a wireless signal to the contact lens 602, receiving a response signal from the contact lens 608, and determining the intraocular pressure of the eye 610. In some embodiments, the method 600 can include additional activities between blocks 602 and 608. For example, after the wireless signal is sent to the contact lens, the method 600 can include initiating a condition to transition a test body on the contact lens from a first state to a second state 604 and removing the condition to transition to the test body on the contact lens from the first state to the second state 606. The intraocular pressure of the eye determine in block 606 can include at least one of a relative intraocular eye pressure or an absolute intraocular eye pressure.

At block 602 a wireless signal is sent from an electronic device, such as a vector network analyzer, to the contact lens on the eye. The contact lens and electronic device can be contact lens 100 and electronic device 150, as described herein, with respect to FIGS. 1A, 1B, and 2, and can include a variable capacitance sensor including an electrical oscillator, as described herein. The wireless signal has a signal frequency. In some examples, the electronic device can vary the signal frequency over a predetermined range, thus sending a plurality of wireless signals to the contact lens, each having a different signal frequency.

In some embodiments, the method 600 also includes actuating a micromagnet in the contact lens with a magnetic actuator. Actuation of the micromagnet includes pushing or otherwise forcing the micromagnet towards the eye for at least one of applanation or indentation of the eye. During applanation or indentation, one or more layers of the sensors in the contact lens are mechanically compressed, altering the space between dielectric layers in the sensor in the contact lens. In some embodiments, the method 600 also includes measuring, with an ASIC in the contact lens, the capacitance produced in the sensor when the one or more layers of the sensor are compressed.

In alternative embodiments, method 600 can include initiating a condition to transition a test body on the contact lens from a first state to a second state 604. In some embodiments, initiating the condition to transition the test body includes initiating a condition in order to transition a test body on a contact lens, for example a test body of a tonometer system, to an expanded state. Accordingly, a contact lens including a test body as described herein, for example with regard to FIGS. 4A-5C can be exposed to a condition such that the test body transitions from an initial state to an expanded state, to thereby exert a force on the eye as shown in FIGS. 5A-5C. For example, where the contact lens includes a tonometer system including a magnetoresponsive test body as described herein, the test body can be exposed to a magnetic field such that the test body transitions from an initial state to an expanded state, for example as shown in FIG. 5B. In some examples, a secondary device, such as an electromagnetic device capable of generating a magnetic field can be positioned near the contact lens to transition the test body from an initial state to an expanded state. For example, a device included in the contact lens can generate the magnetic field. In some examples, the antenna, when it receives a signal carries an electric current that produces a magnetic field, which causes the expandable material to expand. In another example, the user can be provided with a device that generates the magnetic field. In yet another example, the magnetic field is generated by a hand-held device or another type of device. In some examples, the magnetic field is generated in a doctor's office or another type of location.

In some embodiments, initiating the condition to transition the test body includes initiating a condition in order to transition a test body on the contact lens to an expanded state whereby a micromagnet of the test body pushes against or otherwise exerts a force against the eye (such as applanation or indentation). For example, the test body can be exposed to a magnetic field such that the test body transitions from an initial state, whereby the micromagnet of the test body is not exerting a force against the eye, to an expanded state, whereby the micromagnet of the test body is exerting a force against the eye. In some examples, a secondary device, such as an electromagnetic device capable of generating a magnetic field, can be positioned near the contact lens to transition the micromagnet of the test body from an initial state to an expanded state. For example, a device included in the contact lens can generate the magnetic field. In some examples, the antenna, when it receives a signal, carries an electric current that produces a magnetic field, which causes the micromagnet to transition the test body to an expanded state. In another example, the user can be provided with a device that generates the magnetic field. In yet another example, the magnetic field is generated by a hand-held device or another type of device. In some examples, the magnetic field is generated in a doctor's office or another location.

In these alternative embodiments, method 600 can also include removing the condition to transition to the test body on the contact lens from the first state to the second state 606. Removing the condition to transition to the test body can include stopping the condition so that the test body transitions from the expanded state to the initial state 604. In some examples, the contact lens can be removed from the magnetic field, or a device used to generate a magnetic field can be removed from the vicinity of the contact lens. In some examples, where the magnetic field is generated by an electromagnetic device, the magnetic field can be turned off. In some embodiments, when the condition, such as the magnetic field, is turned off or removed the test body can transition from an expanded state to an initial state, as described herein.

In some embodiments, method 600 can include wirelessly monitoring an amount of time involved for the test body to transition from an expanded state to an initial state. The transition time can be wirelessly monitored by, for example, the sensor 120 as described herein with respect to FIGS. 4A and 4B. In some examples, the amount of time involved for the test body to transition is measured by wirelessly detecting the change or rate of change in the mechanical strain experienced by the contact lens. In some examples, this can be achieved with a variable capacitance sensor, as described herein. For example, in some examples, a first mechanical strain can correspond to the expanded state of the magnetoresponsive test body and a second mechanical strain can correspond to the initial state of the magnetoresponsive test body, and the amount of time between detecting the first mechanical strain and detecting the second mechanical strain can be wirelessly recorded, for example by an electronic device in communication with the sensor. However, in some other examples, the deceleration of the test body as the expandable material transitions from an initial state to an expanded state at block 608 can be wirelessly measured or detected. In some embodiments, the method 600 also includes measuring, with an ASIC in the contact lens, the capacitance produced in the sensor when the one or more layers of the sensor are no longer compressed as the test body transitions from the expanded state to the initial state.

At block 608, the electronic device receives a response signal including a measurable characteristic from the contact lens. The measurable characteristics received in the response signal can vary according to different embodiments described herein. In some embodiments, the response signal is sent or transmitted from the contact lens to the electronic device when the signal frequency of the wireless signal sent in block 602 matches or corresponds to the natural frequency of the electrical oscillator. Further, the response signal sent from the contact lens includes a measurable characteristic, such as the frequency of the response signal itself, corresponding to the natural frequency of the electrical oscillator. In some embodiments, the response signal includes a measurable characteristic of a capacitance value measured by the ASIC in the contact lens. In some embodiments, the response signal include a measurable characteristic of the amount of time involved for the test body to transition from the expanded state to the initial state, or the rate of change of the mechanical strain of the contact lens is used to determine the absolute intraocular pressure of the eye. In some examples, where the amount of time for the test body to transition from the expanded state to the initial state can correspond to the absolute intraocular pressure of the eye. Similarly, in some examples, the rate of change of the mechanical strain of the test body can correspond to the absolute intraocular pressure of the eye. In some embodiments, the response signal includes a measurable characteristic of a voltage of the magnetic field of the test body in at least one of the initial state or the expanded state. At block 610 the measurable characteristics of the response signal are used to determine the intraocular pressure of the eye. The intraocular pressure determined by either relative intraocular pressure or absolute intraocular pressure. For example, the natural frequency of the electrical oscillator can be used to determine the relative intraocular pressure of the eye by utilizing, for example, Equation 4 as described herein. In some embodiments, the capacitance measured by the ASIC on the contact lens can be used to determine the relative intraocular pressure of the eye by utilizing, for example, a correlation between the eye pressure and the measured natural frequency. In some embodiments, the absolute intraocular pressure of the eye can be determined from the amount of time involved for the test body to transition from the expanded state to the initial state. In some embodiments, the absolute intraocular pressure of the eye can be determined from the voltage of the magnetic field of the test body in at least one of the initial state or expanded state.

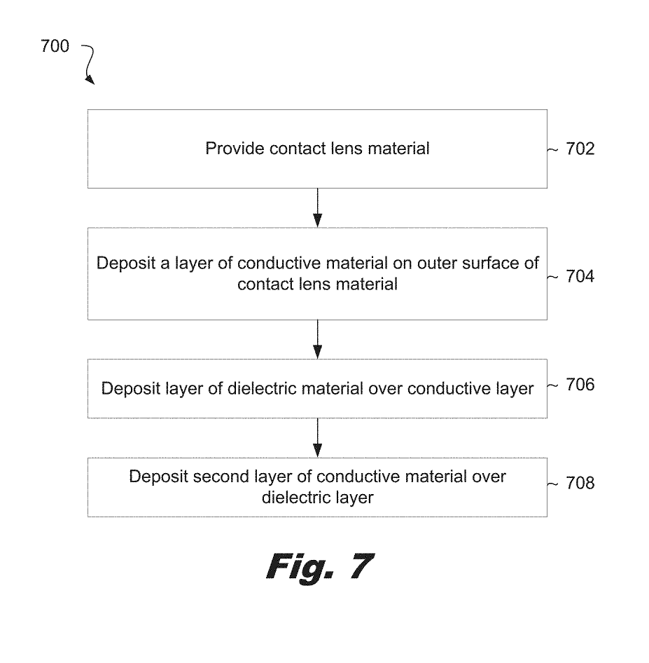

FIG. 7 illustrates an example of a method 700 of manufacturing a sensor on a contact lens for wirelessly monitoring the relative intraocular pressure of an eye. In this example, the method 700 includes providing contact lens material 702, depositing a layer of conductive material on the outer surface of the contact lens 704, depositing a layer of dielectric material over the conductive layer 706, and depositing a second layer of conductive material over the dielectric layer 708, to thereby form the sensor on the contact lens.

At block 702, contact lens material is provided during the manufacturing process. In some examples, the contact lens material can be substantially similar to the contact lens material 110 described above with respect to FIGS. 1A-B, and can include, for example, a silicon hydrogel lens material. In some examples, providing a contact lens can further include depositing or forming an initial substrate layer on which further layers can be deposited. In some examples, this substrate layer can include a layer of dielectric material and can be substantially similar to dielectric layer 122, as described herein with reference to FIGS. 1A-B. Additionally, the substrate layer can be treated, for example to enhance the adhesion or wetting of subsequently deposited layers. In some examples, the substrate layer can be treated by exposure to a plasma for a predetermined period of time. In some other examples, however, the contact lens is provided without additional processing.

At block 704, a layer of conductive material is deposited or formed over the outer surface of the contact lens material provided at block 502. In some examples, the conductive layer can be deposited directly on the contact lens material, however in some other examples, the conductive layer can be deposited on a substrate layer present on the surface of the contact lens material, for example, a substrate layer including dielectric material. The conductive layer can be substantially similar to conductive layer 124, described herein with reference to FIGS. 1A-B.

The conductive layer can be deposited or formed by a variety of methods known in the art, or those that can be developed in the future. In some examples, the conductive layer can be deposited by a spin casting, vapor deposition, or casting molding process. In some examples, the conductive layer can be deposited by a dip coating or deposition process. For example, the conductive layer can include a transparent conductive polymer as described herein and can be deposited by a spin casting process including spinning the contact lens at about 5000 to about 6000 revolutions per minute (RPM). A solution including the transparent conductive material is then applied to the spinning contact lens.

In some examples, after depositing the conductive layer, the contact lens can optionally be treated to cure the conductive layer and/or to allow for deposition of further layers. The treatment process can include heating the contact lens and conductive layer and/or applying a chemical treatment to the conductive layer. For example, after applying the conductive layer to the contact lens, the contact lens can be heated to from about 80° C. to about 120° C. In some examples, the contact lens can be kept at an elevated temperature for from about 15 minutes to about 1 hour, for example about 30 minutes. In some examples, a chemical treatment can be applied to the conductive layer in a treatment process in order to enhance the conductivity of the layer or to improve deposition of subsequent layers. For example, formic acid can be applied to the conductive layer. The formic acid can straighten the polymer chains of the transparent conductive material and thereby increase the conductivity of the layer. In some examples, any chemical treatment applied to the conductive layer can be rinsed or washed away, for example with water, after a predetermined time period. In some examples, the chemical treatment can be washed away after about 1 minute to about 15 minutes, for example about 5 minutes.

At block 706 a dielectric material is deposited or formed over the conductive layer deposited at block 704. The dielectric layer can be substantially similar to dielectric layer 126 described herein with reference to FIGS. 1A-B and can include a transparent polymer material. The dielectric layer can be deposited or formed by a variety of methods as are known in the art or can be developed in the future. In some examples, the dielectric layer can be deposited by a spin or dip coating process that is similar to the deposition process for the conductive layer at block 704. Similarly, the dielectric layer can be subjected to treatment processes, such as curing, as described with respect to block 704 and/or can be exposed to plasma for a predetermined period of time.

At block 708 a second layer of conductive material is deposited or formed on the dielectric layer deposited at block 706 to thereby form the sensor on the contact lens. The second conductive layer can be substantially similar to the conductive layer 128 described herein with reference to FIGS. 1A-B. Additionally, the second conductive layer can be formed or deposited by a variety of methods as are known in the art or can be developed in the future. In some examples, the conductive layer can be deposited by a spin or dip coating process that is similar to the deposition process for the conductive layer at block 704. Similarly, the conductive layer can be subjected to treatment processes, such as curing, as described with respect to block 704 and/or can be exposed to plasma for a predetermined period of time.

The contact lens and sensor can be subjected to additional processing. For example, one or more additional layers of material can be deposited over the sensor, and/or the sensor and contact lens can be subjected to additional curing or treatment processes. In some examples, an antenna structure can be printed or deposited on the topmost conductive layer as described herein. In some examples, an encapsulation layer substantially similar to encapsulation layer 130 described herein with reference to FIGS. 1A-B can be deposited or formed over the sensor.

In some embodiments, a first layer of dielectric material is deposited over the outer surface of the contact lens material, and the first layer of conductive material is deposited over the first layer of dielectric material. The second layer of dielectric material can be deposited over the first layer of conductive material, similar to block 706, and a second layer of conductive material can be deposited over the second layer of dielectric material, similar to block 708.

In these and other embodiments, the method 700 also includes depositing at least a micromagnet over or at least partially within the first layer of the dielectric material or the second layer of the dielectric material. For example, method 700 can include depositing a micromagnet over the first layer of the dielectric material. The micromagnet deposited over the first layer of the dielectric material can be at least partially deposited or embedded within the first layer of conductive material. In other embodiments, the method 700 includes depositing the micromagnet over the second layer of the dielectric material. The micromagnet deposited over the second layer of the dielectric material can be at least partially deposited or embedded within the second layer of conducted material.

In some embodiments, the method 700 also includes depositing a micromagnet over or at least partially within the first layer of the dielectric material or the second layer of the dielectric material. For example, method 700 can include depositing a micromagnet over the first layer of the dielectric material. The micromagnet deposited over the first layer of the dielectric material can be at least partially deposited or embedded within the first layer of conductive material. In other embodiments, the method 700 includes depositing the micromagnet over the second layer of the dielectric material. The micromagnet deposited over the second layer of the dielectric material can be at least partially deposited or embedded within the second layer of conducted material.

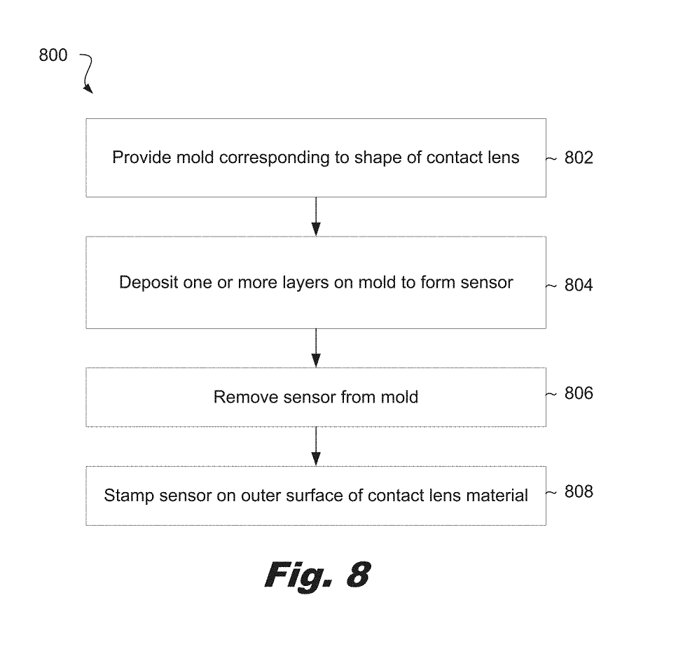

FIG. 8 illustrates an example of a method 800 of manufacturing a sensor on a contact lens for wirelessly monitoring the relative intraocular pressure of an eye. In this example, the method 800 includes providing a mold corresponding to the shape of a contact lens 802, depositing one or more layers on the mold to form the sensor 804, removing the sensor from the mold 806, and applying the sensor to the lens 808 or a partially cured or formed lens.

At block 802, a mold is provided that has a shape corresponding to the shape of the contact lens onto which the sensor will ultimately be deposited. Preferably the mold includes a material having a relatively low surface energy in order to allow for low adhesion and release of the materials which are deposited on the mold. In some examples, the mold can include a polymer material, however, in some other examples, the mold can include a metal or ceramic material. For example, in some examples, the mold can include polytetrafluoroethylene (PTFE).

At block 604 one or more layers are deposited or formed on the surface of the mold to thereby form the sensor. In some examples, the deposited layers can be substantially similar to layers 124, 126, 128 described above with reference to FIGS. 1A-B. Further, in some examples, the layers can be deposited according to the processes described herein, with reference to blocks 704, 706, and 708 of FIG. 7. Thus, in some examples, at least first and second conductive layers, and one or more dielectric layers disposed there between, are deposited on the mold to thereby form the sensor.

At block 806 the sensor is removed or lifted from the mold. In some examples, the sensor can be removed from the mold by a separate tool that can attach or adhere to the sensor. In some examples, this tool can include an adhesive surface and can include silicone gel. The tool should have a surface that has stronger adhesion to the sensor than the sensor has to the mold material to thereby enable clean and easy removal from the mold. The sensor can then be retained on the tool, also referred to as a stamp, until it is ready for delivery to the contact lens.

At block 808, the sensor is stamped or applied onto a contact lens. For example, the tool or stamp described above can be pressed against the contact lens for a predetermined period of time, such as about one to ten seconds, such that the sensor adheres to the outer surface of the contact lens. The stamp can then be removed from the contact lens, leaving the sensor adhered or bonded to the surface of the contact lens. The contact lens can be substantially similar to the lens 110 described herein with reference to FIG. 1A-B. Further, the contact lens can have an additional substrate layer deposited thereon, and can be subjected to treatments, such as exposure to plasma as described herein, with reference to block 702 of FIG. 7.

### EXAMPLE

An example variable capacitance sensor was manufactured by a dip and spin casting process, as described herein, and according to some embodiments. A silicone hydrogel contact lens was provided and a first transparent dielectric polymer layer was deposited thereon to serve as a substrate layer. A solution of PDMS was dropped onto the outer surface of the lens at room temperature. Excess PDMS was drained from the sides of the contact lens and the lens was allowed to rest at room temperature until a thin layer of PDMS was left. The PDMS layer was exposed to a plasma in order to enhance the wetting properties of the subsequently applied layers.

A solution of PEDOT:PSS was applied to the PDMS dielectric layer by a spin casting process, with the lens being spun at about 5000 to 6000 RPM. After applying the PEDOT:PSS solution, the lens was heated in an oven to 100° C. for 30 minutes to form a transparent conductive layer. A drop of 45% by weight formic acid was then applied to the cured conductive layer and was allowed to remain on the lens for 5 minutes, before being washed away. The thickness of this conductive layer was measured and found to be less than about 5 microns.

A PDMS dielectric layer was applied to the conductive layer by a similar process to the deposition of the substrate layer, and a second PEDOT:PSS conductive layer was deposited over the dielectric layer to form the variable capacitance sensor.

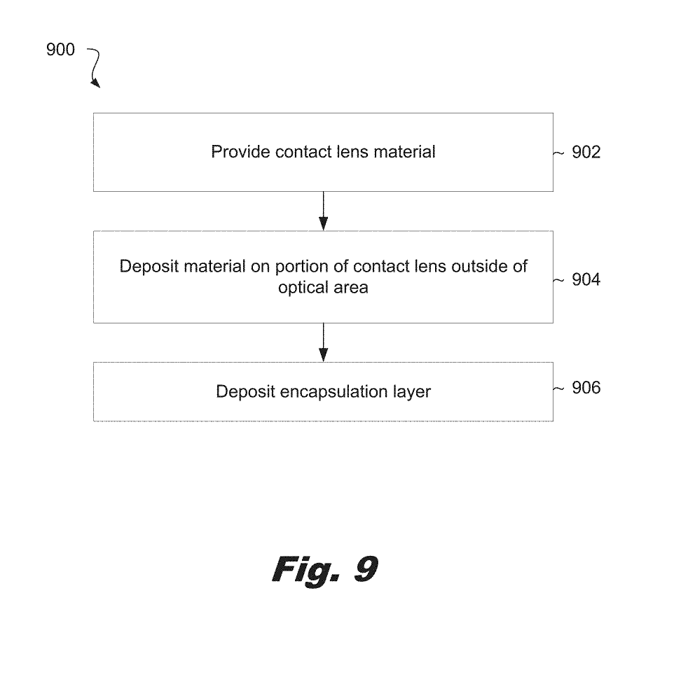

FIG. 9 illustrates a method 900 of manufacturing a test body, for example as a component of a tonometer system, on a contact lens for wirelessly determining the intraocular pressure of an eye. In this example, the method 900 includes providing a contact lens material 902, depositing at least a layer of material to form the test body 904, and depositing an encapsulation layer over the test body and contact lens material 906.

At block 902, contact lens material is provided during the manufacturing process. In some examples, the contact lens material can be substantially similar to the contact lens material 110 described herein with respect to FIGS. 4A and 4B. For example, the contact lens material can include a silicone hydrogel material. However, in some examples, providing the contact lens material can include depositing or forming an additional substrate layer or layers on the contact lens for the subsequently deposited test body to adhere to. For example, in some examples, a polymer layer, such as a polydimethylsiloxane (PDMS) layer can be deposited on the contact lens material. Additionally, the substrate layer can be treated, for example, to enhance the adhesion or wetting of subsequently deposited layers. In some examples, the substrate layer can be treated by exposure to a plasma for a predetermined period of time. In some other examples, however, the contact lens is provided without additional processing.

At block 904, a material is deposited on a portion of the contact lens outside of the optic zone to form the test body. In some embodiments, the material includes an expandable material. For example, the expandable material can include a magnetoresponsive elastomer, as described herein. In some embodiments, the expandable material can be deposited by printing, for example, inkjet printing, the expandable material on the desired portion of the contact lens. In some other examples, the expandable material can be deposited on a portion of the contact lens outside of the optic area by spin casting or dip coating. In some examples, an additional layer or layers can further be deposited to form the test body. Further, in some other embodiments, a sensor, such as sensor 120 described herein with respect to FIGS. 4A and 4B can be deposited at block 904. In some examples, the sensor can be deposited or formed prior to forming the test body, however, in some other examples, the sensor can be deposited or formed after depositing the test body. Block 904 can also include curing or treating the deposited expandable material to thereby for the test body, for example by exposure to curing agents, heat, and/or plasma, as is known in the art.

In some embodiments, depositing a material on a portion of the contact lens outside the optical area includes depositing at least a micromagnet on a portion of the contact lens outside the optical area. Depositing at least the micromagnet on the portion of the contact lens outside the optical area can include depositing at least the micromagnet on the contact lens with the sensor deposited on the contact lens. In some embodiments, depositing at least a micromagnet on the contact lens outside the optical area includes depositing inserting a thin ferromagnetic disc in the contact lens mold, then magnetizing the ferromagnetic disc in a desired direction after polymerization of the contact lens. Alternatively, the method can include using a magnetized disc to position the micromagnet within the contact lens during polymerization.

In some embodiments, depositing at least the micromagnet on the portion of the contact lens outside the optical area includes depositing a micromagnet array on the portion of the contact lens outside the optical area. More particularly, multiple layers of micromagnet arrays can be deposited on the portion of the contact lens outside the optical area. In certain embodiments, depositing the micromagnet array on the portion of the contact lens outside the optical area can include sprinkling or otherwise depositing hard magnetic particles onto a mold (or other master structure), then pouring a contact lens monomer structure in the mold, and finally peeling the contact lens including the magnetic particles off the mold. In some embodiments, the magnetic particles sprinkled or deposited on the mold can be patterned into one or more predetermined positions on the mold through, for example, one or more magnets or other materials positioned in the mold to direct the magnetic particles to the one or more predetermined positions.

In some embodiments, depositing a material on a portion of the contact lens outside the optical area includes coating the test body with a contrast agent configured to enable time-of-flight proximity sensing. For example, coating the test body with the contrast agent can include coating the test body with at least one of a radiation source layer, a dye, or a tag. Alternatively, the method can include coating a surface of the test body to modify reflectance and enable time-of-flight proximity. The surface of the test body can also include a selective specific transmitter time-of-flight system for position measurement. In these and other embodiments, the radiation source layer can be replaced by a radiation reflector material.

At block 906, an encapsulation layer can be deposited on the contact lens over the test body. The encapsulation layer can be substantially similar to encapsulation layer 130 described herein with reference to FIGS. 4A and 4B. For example, in some examples, the encapsulation layer can be a hydrogel material, such as a silicone hydrogel material. In some examples, the encapsulation layer can be the same material as the contact lens material. The encapsulation layer can be deposited on the contact lens, for example by printing, spin casting, cast molding, or dip coating.

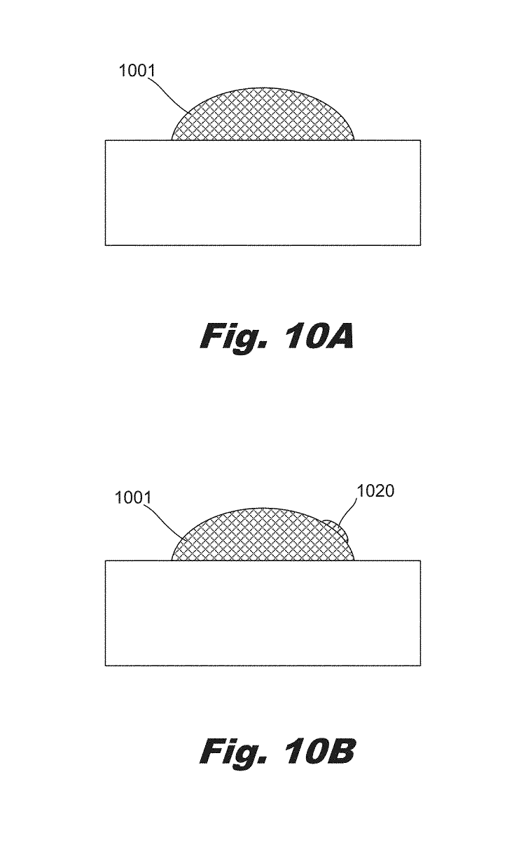

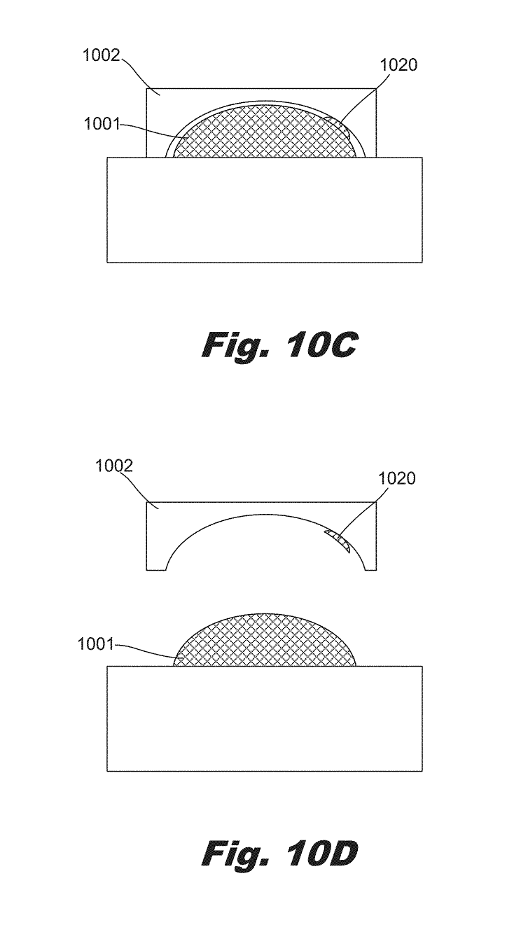

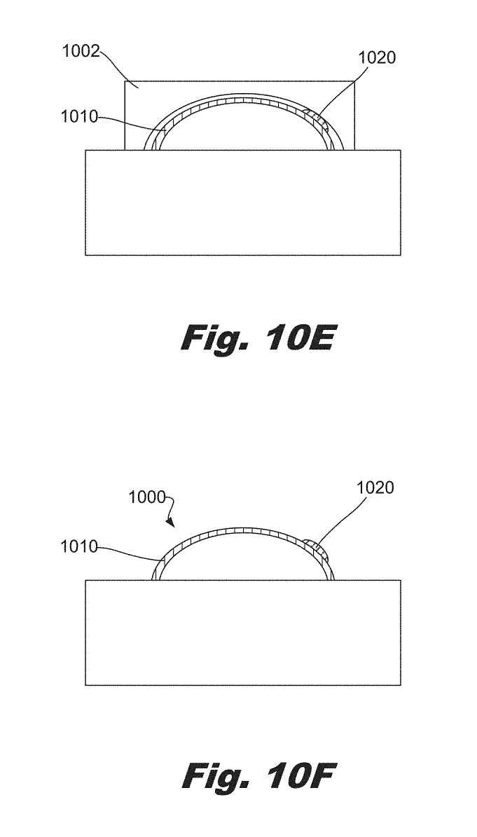

In some other embodiments, however, a test body on a contact lens for wirelessly determining the intraocular pressure of an eye can be manufactured by a stamping process or method as illustrated in FIGS. 10A-10F. In this example, the method includes providing a mold 1001 corresponding to the shape of a contact lens material 1010, depositing one or more layers on the mold to form the test body 1020, removing the test body 1020 from the mold 1001 with a stamping tool 1002, and applying the test body 1020 to the lens material 1010 to thereby form the contact lens 1000.

As illustrated in FIG. 10A, a mold 1001 is provided that has a shape corresponding to the shape of the contact lens material 1010 onto which the test body 1020 will ultimately be deposited. The mold 1001 can include a material having a relatively low surface energy to allow for low adhesion and release of the material deposited on the mold 1001. In some examples, the mold 1001 can include a polymer material, however in some other examples, the mold 1001 can include a metal or a ceramic material. For example, the mold 1001 can include polytetrafluoroethylene (PTFE).

As illustrated in FIG. 10B, one or more layers can then be deposited or formed on the surface of the mold 1001, to thereby form the test body 1020 as described herein. In some examples, the deposited material can include at least a micromagnet. In some examples, the deposited layer can include at least an expandable material, such as a magnetoresponsive hydrogel elastomer, as described herein. Further, in some examples, the test body can be deposited or formed in a substantially similar manner to the processes described herein with reference to FIG. 9, except that deposition occurs on the mold 1001, rather than directly on the lens material as in FIG. 9. In some examples, at least a layer including an expandable material is deposited or formed on the mold 1001 by, for example, printing, spin casting, or dip coating to form the test body 1020.

As illustrated in FIGS. 10C-10D, the test body 1020 can be removed or lifted from the mold 1001. In some examples, the test body 1020 can be removed from the mold by a separate tool 1002 that can attach or adhere to the test body 1020. In some examples, the tool 1002 can have an adhesive surface and can include silicone gel. The tool 1002 should have a surface that has stronger adhesion to the test body 1020 than the test body 1020 has to the mold material 1001, to thereby enable clean and easy removal from the mold 1001. The test body 1020 can then be retained on the tool 1002, also referred to as a stamp, until it is ready for delivery to the contact lens material 1010.

As illustrated in FIG. 10E, the test body 1020 is stamped or applied into or onto contact lens material 1010. The contact lens material 1010 can be substantially similar to the contact lens material 110 described herein, with reference to FIGS. 4A and 4B. Advantageously, the stamping process described herein allows for parallel manufacturing of the test body 1020 and the lens material 1010, thereby allowing for increased producing speed. In some examples, the tool or stamp 1002 can be pressed against the contact lens material 1010 for a predetermined period of time, such as about one to ten seconds, such that the test body 1020 adheres to the outer surface of the contact lens material 1020.

As illustrated in FIG. 10F, the stamp 1002 can then be removed, leaving the test body 1020 adhered or bonded to the surface of the contact lens material 1010, thereby forming the contact lens 1000. The contact lens can be substantially similar to the contact lens 400 described herein with reference to FIGS. 4A and 4B. Further, the contact lens 1000 can have an additional substrate layer deposited thereon, such as an encapsulation layer as described herein. The contact lens 1000 can also be subject to further processing and can be subjected to treatments, such as heating, exposure to treatment chemicals, and/or plasma.

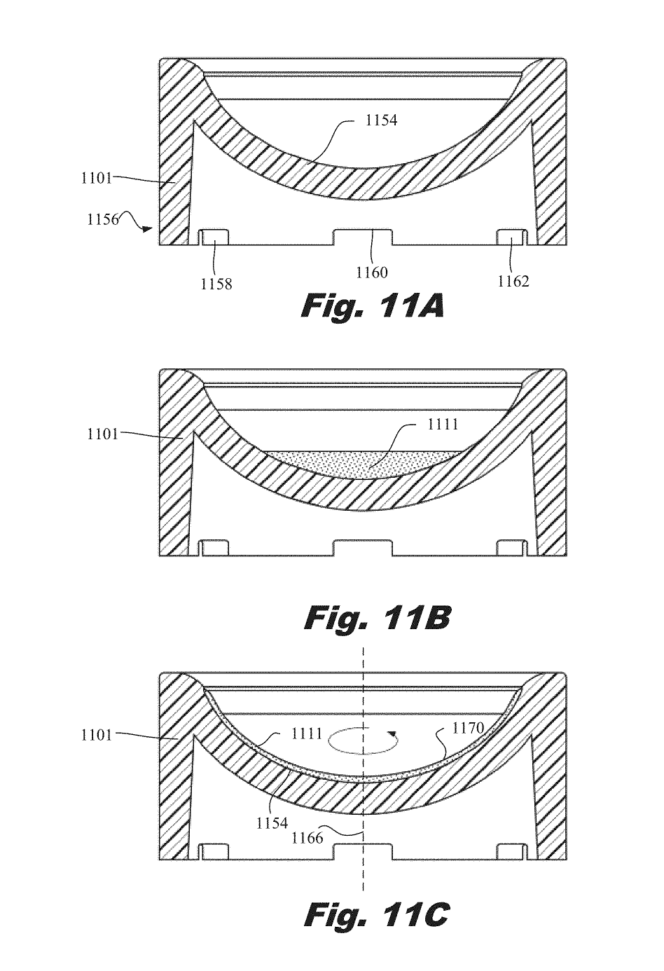

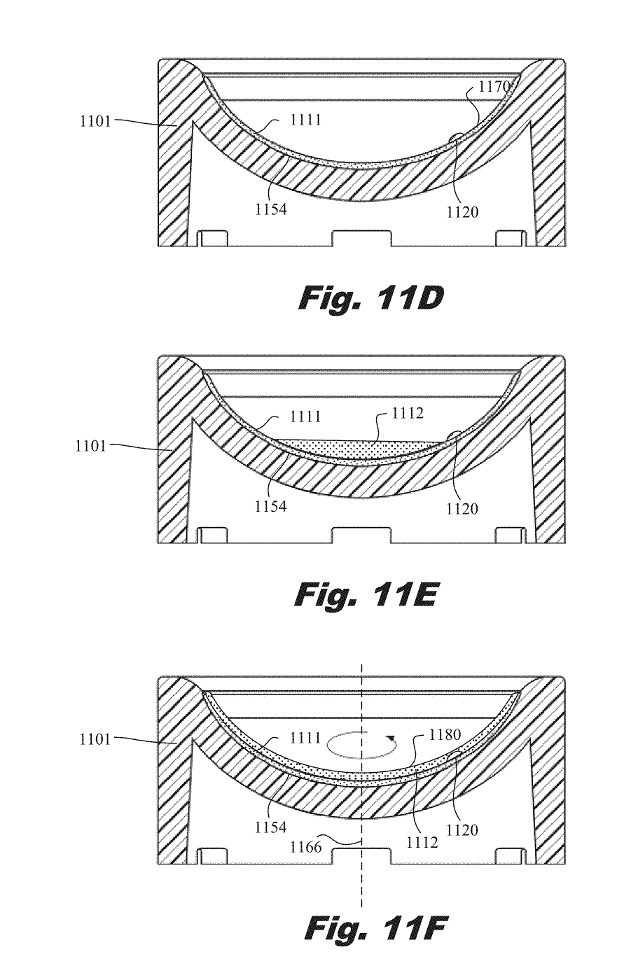

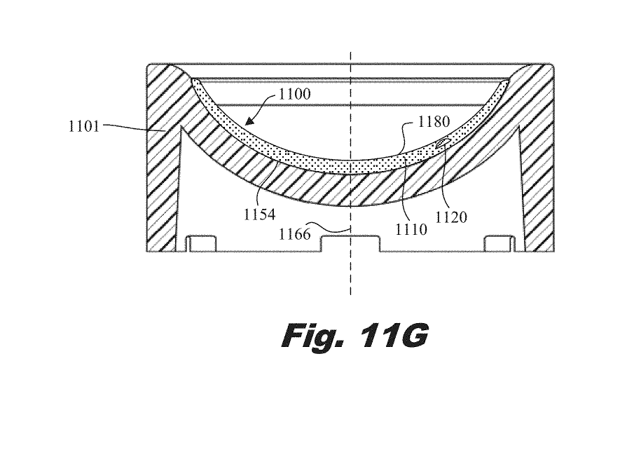

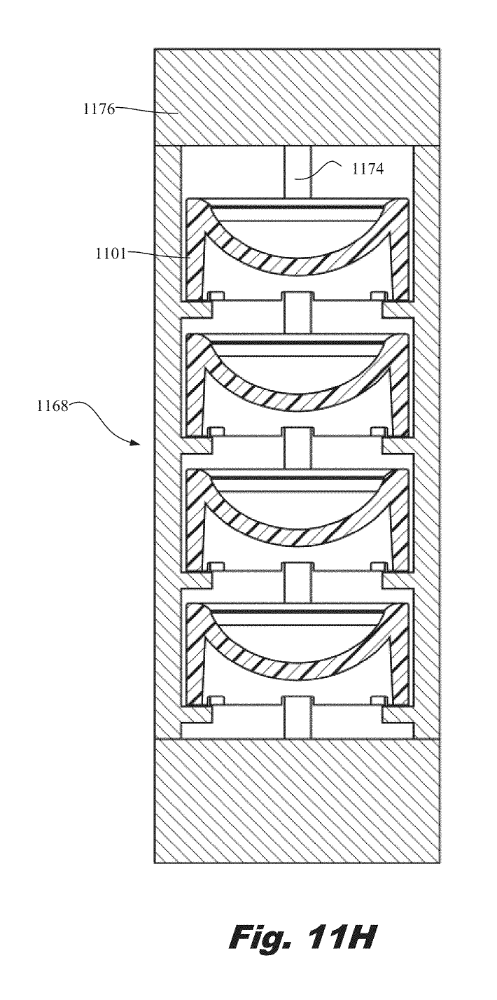

FIGS. 11A-11H illustrate various components that can be used in certain examples for making a contact lens 1100, in accordance with the principles described in the present disclosure. A liquid lens material 1111 can be applied to a profile 1154 of the mold 1101. The mold 1101 with the liquid lens material 1111 can be loaded into a spinning structure 1168 that is configured to spin the mold 1101 so that the liquid lens material 1111 centrifugally spreads across the profile 1154 into the desired shape of the front surface of the contact lens 1100. A curing agent (e.g., temperature, actinic radiation, or another type of curing agent) can be exposed to the liquid lens material 1111 while the mold 1101 is spinning. In some examples, no curing agent is used. As a result of the curing agent, or merely the elapse of time, the liquid lens material 1111 can assume a gel state having the desired profile of the contact lens 1100. A test body 1120 can then be stamped, formed, or otherwise deposited on the posterior surface 1170 of the lens material 1111. A second liquid lens material 1112 can be applied to the posterior surface 1170 of the liquid lens material 1111, whereupon the mold 1110 with the liquid lens material 1111, test body 1120, and second liquid lens material 1112 is spun so that the second liquid lens material 1112 centrifugally spreads across the posterior surface 1170 and envelops or surrounds the test body 1120. A curing agent (e.g., temperature, actinic radiation, or another type of curing agent) can be exposed to the liquid lens materials 1111, 1112 while the mold 1101 is spinning. In some examples, no curing agent is used. As a result of the curing agent, or merely the elapse of time, the liquid lens materials 1111, 1112 can form contact lens 1100.

FIG. 11A is a cross-sectional view of one embodiment of a mold for a contact lens, according to the principles of the present disclosure. In this example, the mold 1101 has a base 1156 with multiple cut outs 1158, 1160, 1162 that are spaced and shaped to interlock with an internal surface of a spinning structure during a later stage of manufacturing. The profile 1154 of the mold 1101 is shaped to form the anterior surface of the contact lens 1100. In some examples, the profile 1154 of the mold 1101 can be continuous without substantial interruptions.

FIG. 11B is a cross-sectional view of one embodiment of a mold 1101 with a liquid lens material 1111, according to the principles of the present disclosure. In this example, the liquid lens material 1111 is deposited into the profile 1154 of the mold.

The liquid lens material 1111 can be made from any material suitable for use in contact lenses. For example, the liquid lens material 1111 can be made of any silicone material and/or hydrogel material. Such material can be formed of polymers, such as tefilcon, tetrafilcon A, crofilcon, helfilcon A&B, mafilcon, polymacon, hioxifilcon B, lotrafilcon A, lotrafilcon B, galyfilcon A, senofilcon A, sifilcon A, comfilcon A, enfilcon A, lidofilcon B, surfilcon A, lidofilcon A, alfafilcon A, omafilcon A, vasurfilcon A, hioxifilcon A, hioxifilcon D, nelfilcon A, hilafilcon A, acofilcon A, bufilcon A, deltafilcon A, phemfilcon A, bufilcon A, perfilcon, etafilcon A, focofilcon A, ocufilcon B, ocufilcon C, ocufilcon D ocufilcon E, ocufilcon F, phemfilcon A, methafilcon A, methafilcon B, vilfilcon A, other types of polymers, monomers, or combinations thereof. These materials can include various combinations of monomers, polymers, and other materials to form the liquid lens material.

In one embodiment, the liquid lens material 1111 is made of hydrogel polymers without any silicone. This can be desirable to increase the wettability of the contact lens. In another embodiment, the liquid lens material 1111 is made of silicone hydrogel material.

FIGS. 11B and 11C are cross-sectional views of a mold 1101 with a liquid lens material 1111 centrifugally spreading across a profile 1154 of the mold 1101, according to the principles of the present disclosure. In this example, the mold 1101 is spun around a central axis 1166 within a spinning structure (1168, FIG. 11H). The spinning structure 1168 is rotated at a speed and in such a way that forms the desired posterior surface 1170 of the liquid lens material 1111 in a gel state.

The spinning structure 1168 includes a central loading region that can receive the molds 1101 that contain the liquid lens material 1152. The central loading region can be formed by a glass tube, a metal tube, or another type of structure that can retain the molds 1101 in a stacked orientation. In examples where actinic radiation is used as the curing agent, the spinning structure 1168 can have an opaque material, a semi-transparent material, or a transparent material that include a sufficient amount of openings to allow the actinic radiation into the central loading region. In the example of FIG. 11H, the spinning structure 1168 includes multiple guide posts 1174 that retain the molds 1101 in a stacked orientation. The spinning structure 1168 also includes a region 1176 that can be used to attach to a spinning driver, such as a motor.

The spinning structure 1168 can be programmed to rotate in a precise manner to form the desired posterior surface 1170 of the gel state liquid lens material 1111. The program that causes the spinning structure 1168 to rotate can be modified to create a desired profile for different users based on each user's individual prescription. The curing agent can be applied to the liquid lens material 1152 while the spinning structure 1168 rotates the molds 1101. As a result, gel state liquid lens material 1111 is formed while the spinning structure rotates. In some examples, the gel state liquid lens material 1111 is fully cured within the spinning structure. But, in other examples, the gel state liquid lens material 1111 can be fully cured over the course of multiple curing stages. For example, the gel state liquid lens material 1111 can be cured in the spinning structure 1168 to a point where the liquid lens material retains its shape, but is not fully cured.

At this stage, a test body 1120 is stamped, formed, or otherwise deposited on the posterior surface 1170 of the gel state liquid lens material 1111. The test body 1120 can be stamped, formed, or otherwise deposited by any of the methods as described herein. The test body can be substantially similar to test body 125 described herein. In some examples, a tool or stamp can be pressed against the gel state liquid lens material 1111 for a predetermined period of time, such that the test body 1120 adheres to the posterior surface 1170 of the gel state liquid lens material 1111.

A second liquid lens material 1112 can be deposited in the mold 1101 over the gel state liquid lens material 1111. In some embodiments the second liquid lens material 1112 can be made from any material suitable for use in contact lenses. For example, the second liquid lens material 1112 can be any of the materials described herein with reference to liquid lens material 1111. In some examples, the second liquid lens material 1112 can be the same material as liquid lens material 1111. However, in some other embodiments the second liquid lens material 1112 can include a different material than the liquid lens material 1111.

FIGS. 11E and 11F are cross-sectional views of a mold 1101 with a second liquid lens material 1112 centrifugally spreading across a posterior surface 1170 of the gel state liquid lens material 1111 and enveloping a test body 1120, according to the principles of the present disclosure. In this example, the mold 1101 is spun around a central axis 1166 within a spinning structure (1168, FIG. 11H). The spinning structure 1168 is rotated at a speed and in such a way that envelops the test body 1120 and forms the desired posterior surface 1180 of the contact lens 1100, for example, in a similar manner as described herein with respect to FIGS. 11B and 11C.

The spinning structure 1168 can be programmed to rotate in a precise manner to form the desired posterior surface 1180 of the contact lens 1100. The program that causes the spinning structure 1168 to rotate can be modified to create a desired profile for different users, based on each user's individual prescription. The curing agent can be applied to the first and/or second liquid lens materials 1111, 1112 while the spinning structure 1168 rotates the molds 1101. As a result, the contact lens 1100 is formed while the spinning structure rotates. In some examples, the liquid lens materials 1111, 1112 are fully cured within the spinning structure. In other examples, however, the liquid lens materials 1111, 1112 can be fully cured over the course of multiple curing stages. In some embodiments, the cured contact lens 1100 can include two separate layers, each formed of their respective liquid lens materials 1111, 1112. However, in some other embodiments, the cured contact lens 1100 can include a single continuous layer or portion of material formed from liquid lens materials 1111, 1112.

FIG. 11G illustrates a cured contact lens 1100 formed from liquid lens materials 1111, 1112 and including the test body 1120 incorporated therein. At this stage, the mold 1101 with the contact lens 1100 can be removed from the spinning structure to finish curing in an environment that is cost effective. A spinning structure that is compatible with the principles described herein is described in U.S. Patent Publication 2012/0133064 issued to Stephen D. Newman. U.S. Patent Publication 2012/0133064, which application is incorporated herein by reference, for all that it discloses.

The contact lens 1100 can be shaped and sized based on a variety of factors, including the shape and size of the user's eye and various optical properties to be achieved by a central optical portion of the contact lens. In some examples, the total thickness of the contact lens 1100 can be approximately 0.1 mm to approximately 0.14 mm. The thickness of the contact lens 1100 can gradually vary at different locations on the contact lens 1100. For example, the contact lens 1100 can be thicker near the outer edge of the contact lens 1100 relative to the central portion of the contact lens 1100.

In one embodiment, the contact lens includes a lens material, and a sensor coupled to the lens material, wherein the sensor is configured to sense a measurable characteristic used to determine an intraocular pressure of an eye to which the contact lens is applied.

In one embodiment, the sensor includes a variable capacitance sensor configured to change capacitance in response to a mechanical strain of the lens material, and the intraocular pressure of the eye is proportional to the mechanical strain of the lens material.

The contact lens further includes an outer surface, and the sensor includes a layer of a conductive material disposed over the outer surface of the contact lens, and a layer of a dielectric material overlying the conductive material.

The conductive material can include a transparent polymer material including poly(3,4-ethylenedioxythiophene) polystyrene sulfonate.

In one embodiment, the layer of the conductive material includes a continuous layer. In one embodiment, the layer of the conductive material has a thickness of from about 1 micrometer to about 10 micrometers.

In one embodiment, the dielectric material includes a polymer material. In another embodiment, the dielectric material includes a silicone material. In one embodiment, the dielectric material includes polydimethylsiloxane. In one embodiment, the dielectric material is a continuous layer.

According to one embodiment, the layer of the conductive material has a thickness of from about 1 micrometer to about 10 micrometers.

In yet another embodiment, the sensor further includes a second layer of conductive material overlying the layer of the dielectric material. In one embodiment, the sensor includes a parallel plate capacitor. A capacitance of the parallel plate capacitor can correspond to a mechanical strain of the lens material. In one embodiment, the sensor comprises an electrical oscillator having a frequency corresponding to the strain of the lens material.

Additionally, the contact lens can include an antenna structure forming a part of the sensor. In one embodiment, the second conductive material includes the antenna structure.

In one embodiment, the frequency of the electrical oscillator corresponds to a stretch factor of the contact lens from an initial state on an eye according to the equation:

$$f = \frac{1}{2 π {λ}^{2} \sqrt{{LC}_{0}}}$$

wherein f is the frequency of the electrical oscillator, λ is the factor of how much the contact lens has stretched on the eye, L is an inductance of the antenna structure, and C₀ is a capacitance of the capacitor when the contact lens is in the initial state. In one embodiment, a mechanical strain of the contact lens is proportional to λ, and a relative intraocular pressure of the eye is proportional to λ.

In one embodiment, the sensor of the exemplary contact lens includes a variable capacitance sensor having a capacitor including a first layer of a conductive material, a second layer of the conductive material, and a layer of a dielectric disposed between the first and second layers of conductive material, and an antenna having an inductance and electrically connected to the capacitor, the second layer of the conductive material including the antenna. In one exemplary embodiment, the sensor has a thickness of from about 20 micrometers to about 50 micrometers.

In one embodiment, the lens material includes an outer surface, the lens further including an encapsulation layer, and the sensor being positioned between the lens material and the encapsulation layer, the sensor including: a first layer of dielectric material disposed over the outer surface of the lens material, a first layer of conductive material disposed over the first layer of the dielectric material, and a micromagnet configured to exert a force on at least the first layer of dielectric material and the first layer of conductive material responsive to actuation, the force exerted by the micromagnet on at least the first layer of dielectric material and the first layer of conductive material responsive to actuation configured to applanate or indent a portion of the eye.

In one embodiment, the sensor further includes an application specific integrated circuit configured to measure capacitance of the sensor as the micromagnet exerts the force on at least the first layer of dielectric material and the first layer of conductive material.

In one embodiment, the capacitance is used to determine a frequency corresponding to the strain of the lens material, the frequency being approximately 4.1395 Hz.

In one embodiment, the sensor includes a second layer of dielectric material disposed over the first layer of conductive material, and a second layer of conductive material disposed over the second layer of the dielectric material, wherein the micromagnet is configured to exert the force on at least the first layer of dielectric material, the first layer of conductive material responsive to actuation, and the second layer of the dielectric material.

In one embodiment, the sensor is responsive to a magnetic actuator configured to actuate the micromagnet.

In one embodiment, the sensor further includes a second layer of dielectric material disposed over the first layer of conductive material, and a second layer of conductive material disposed over the second layer of the dielectric material, an application specific integrated circuit configured to measure capacitance of the sensor as the micromagnet exerts the force on at least the first layer of dielectric material, the first layer of conductive material, and the second layer of dielectric material, and wherein, responsive to actuation from a magnetic actuator, the micromagnet is configured to exert the force on at least the first layer of dielectric material, the first layer of conductive material responsive to actuation, and the second layer of the dielectric material.

In one exemplary embodiment, the contact lens includes a tonometer system formed in the contact lens and including the sensor, and a test body in contact with the lens material configured to exert a force on an eye in the presence of a magnetic field when the contact lens is positioned on the eye. In one embodiment, the test body includes an expandable material and the sensor is in contact with the lens material, the force exerted on the eye causes a mechanical strain in the contact lens, and the sensor detects a rate of change of the mechanical strain of the contact lens when the force is no longer exerted on the eye. In one embodiment, the rate of change of the mechanical strain of the contact lens upon release of the force of the expandable material corresponds to an absolute intraocular pressure of the eye.

In one embodiment, the test body includes at least one of an expandable polymer material, an expandable magnetoresponsive hydrogel material, or an expandable magnetoresponsive elastomer material.

In one embodiment, the expandable material expands by the alignment of polymeric microchains in the material. In one embodiment, the expandable material is disposed within the lens material outside an optic zone of the contact lens. In one embodiment, the test body is disposed on an outer surface of the contact lens and outside and optic zone of the contact lens. In one embodiment, the test body has a diameter of from about 1 millimeters to about 3 millimeters when not in the presence of a magnetic field.

In one embodiment, the test body has a thickness of from about 50 micrometers to about 100 micrometers.

In one embodiment, the sensor includes a parallel plate capacitor having a first layer and a second layer of a conductive material and a layer of a dielectric material disposed between the first layer and the second layer of conductive material.

In one embodiment, the sensor further includes an antenna structure.

In one embodiment, the test body includes a micromagnet disc or a micromagnet array configured to exert the force on the eye, the sensor detects a rate of change of the mechanical strain of the contact lens when the force is no longer exerted on the eye, the rate of change of the mechanical strain of the contact lens upon release of the force of the expandable material corresponding to an absolute intraocular pressure of the eye. In one embodiment, the rate of change is measured by a gradiometer. In one embodiment, the rate of change is between 1 and 20 mV.

In another embodiment, the micromagnet disc or micromagnet array are actuated by a magnetic actuator to exert the force on the eye.

In one embodiment, the test body is coated with a contrast agent, the contrast agent including at least one of a radiation source layer, a radiation reflector material, a dye, or a tag.

In one embodiment, a contact lens system for wirelessly determining an intraocular pressure of an eye of a user, includes a contact lens, the contact lens including a lens material having an outer surface and a sensor including one or more transparent polymer layers disposed over the outer surface of the lens material, wherein the sensor is configured to sense measurable characteristic corresponding to the intraocular pressure of the eye of the user, and an electronic device configured to wirelessly receive the measurable characteristic sensed by the sensor.

In one embodiment a method of manufacturing a sensor on a contact lens, includes: providing a contact lens having an outer surface, depositing a layer of a conductive material over the outer surface of the contact lens, and depositing a layer of a dielectric material over the conductive material to form the sensor on the contact lens, wherein the sensor is configured to measure a mechanical strain of the contact lens.

In one embodiment, the method includes depositing a second layer of the conductive material over the layer of the dielectric material. The method alternatively includes depositing an encapsulation layer of the second layer of the conductive material. In on embodiment, the method includes depositing a base layer of the dielectric material directly on the outer surface of the contact lens prior to depositing the layer of the conductive material. The method can further include treating the base layer of the dielectric material to enable the conductive material to wet the base layer prior to depositing the layer of the conductive material thereon.

In one embodiment, depositing the layer of the conductive or dielectric material includes spin casting the conductive or dielectric material on the contact lens. In one embodiment, depositing the layer of the conductive or dielectric material includes stamping the layer of the conductive or dielectric material on the contact lens. According to one exemplary method, the layer of the conductive material and the layer of the dielectric material are simultaneously stamped on the contact lens.

In one embodiment, depositing the layer of the conductive or dielectric material includes stamping the layer of the conductive or dielectric material on the contact lens.

In another embodiment, the method further includes curing at least the deposited layer of conductive material, the deposited layer of dielectric material, and the contact lens. Additionally, in one embodiment, the method further includes depositing at least a micromagnet over the layer of the dielectric material, wherein the micromagnet is configured to exert a pressure on the layer of the dielectric material upon actuation.

One exemplary method of manufacturing a test body on a contact lens includes: providing a mold having a shape corresponding to a shape of a contact lens, depositing a test body structure on an outer surface of the mold, and treating the test body structure or contact lens to adhere the test body structure to the contact lens.

The method in one embodiment, further includes transferring the test body structure from the mold to a stamp, and pressing the stamp against an outer surface of a contact lens to transfer the test body structure from the stamp to the contact lens.

In one embodiment, depositing the test body structure on the outer surface of the mold includes printing one or more layers making up the test body structure. In on embodiment, depositing the test body structure on the outer surface of the mold includes spin casting a layer comprising the test body structure onto the mold.

According to one embodiment, depositing a test body structure on an outer surface of the mold includes depositing a micromagnet disc or a micromagnet array on an outer surface of the mold.

An exemplary method of wirelessly determining an intraocular pressure of an eye includes sending a wireless signal from an electronic device to a contact lens on the eye, the wireless signal having a signal frequency, wherein the contact lens comprises an outer surface and a sensor disposed on the outer surface, receiving a response signal from the contact lens by the electronic device when the signal frequency of the wireless signal matches the natural frequency of the electrical oscillator, the response signal including a measurable characteristic corresponding to the intraocular pressure of the eye, and determining the intraocular pressure of the user from the measurable characteristic of the response signal.

As used herein with reference to contact lenses, the terms ‘top’ or ‘upper’ generally refer directionally to the outer surface of the contact lens when worn as intended by a user, while the terms ‘bottom’ or ‘lower’ generally refer directionally to the inner surface, or eye-facing surface of the lens. Such terms are used for reference and to aid in the understanding of the present disclosure and are not intended to limit the scope of the present disclosure in any way. For example, as used herein, one embodiment of the exemplary lens describes the variable capacitance sensor being formed on the outer top surface of a contact lens. However, the variable capacitance sensor can be formed on the top, bottom, or inner layer of the lens.

Unless otherwise indicated, all numbers or expressions, such as those expressing dimensions, physical characteristics, etc., used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

In addition, all ranges disclosed herein are to be understood to encompass and provide support for claims that recite any and all subranges or any and all individual values subsumed therein. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

We claim:

1. A contact lens, comprising:
a lens material; and
a sensor coupled to the lens material;
wherein the sensor is configured to sense a measurable characteristic used to determine an intraocular pressure of an eye to which the contact lens is applied.

2. The contact lens of claim 1, wherein:
the sensor comprises a variable capacitance sensor configured to change capacitance in response to a mechanical strain of the lens material; and
the intraocular pressure of the eye is measurable relative to the mechanical strain of the lens material.

3. The contact lens of claim 2, wherein:
the contact lens further comprises an outer surface; and
the sensor comprises:
a layer of a conductive material disposed over the outer surface of the contact lens; and
a layer of a dielectric material overlying the conductive material.

4. The contact lens of claim 3, wherein the conductive material is a transparent polymer material comprising poly(3,4-ethylenedioxythiophene) polystyrene sulfonate.

5. The contact lens of claim 3, wherein the layer of the conductive material comprises a continuous layer.

6. The contact lens of claim 3, wherein the layer of the conductive material has a thickness of from about 1 micrometer to about 10 micrometers.

7. The contact lens of claim 3, wherein the dielectric material comprises polydimethylsiloxane.

8. The contact lens of claim 3, the sensor further comprising a second layer of conductive material overlying the layer of the dielectric material.

9. The contact lens of claim 8, wherein the sensor comprises a parallel plate capacitor formed by the two conductive material layers and dielectric material layer.

10. The contact lens of claim 9, wherein the capacitance of the parallel plate capacitor changes with the mechanical strain of the lens material.

11. The contact lens of claim 8, further comprising an antenna structure forming a part of the sensor.

12. The contact lens of claim 11, wherein the sensor comprises an electrical oscillator having a frequency corresponding to the strain of the lens material.

13. The contact lens of claim 12, wherein the frequency of the series or parallel electrical oscillator corresponds to a stretch factor of the contact lens from an initial state on an eye according to the equation:
$$f = \frac{1}{2 π {λ}^{2} \sqrt{{LC}_{0}}}$$
wherein f is the frequency of the electrical oscillator, λ is the factor of how much the contact lens has stretched on the eye, L is an inductance of the antenna structure, and C₀ is a capacitance of the capacitor when the contact lens is in the initial state.

14. The contact lens of claim 13, wherein a mechanical strain of the contact lens is sensitive to λ, and a relative intraocular pressure of the eye is sensitive or correlated to λ.

15. The contact lens of claim 1, wherein the sensor includes a variable capacitance sensor having:
a capacitor comprising a first layer of a conductive material, a second layer of the conductive material, and a layer of a dielectric disposed between the first and second layers of conductive material; and
an antenna having an inductance and electrically connected to the capacitor, the second layer of the conductive material including the antenna.

16. The contact lens of claim 1, wherein the sensor has a thickness of from about 20 micrometers to about 50 micrometers.

17. The contact lens of claim 1, wherein:
the lens material comprises an outer surface;
the contact lens further comprises an encapsulation layer; and
the sensor is positioned between the lens material and the encapsulation layer, the sensor including:
a first layer of dielectric material disposed over the outer surface of the lens material;
a first layer of conductive material disposed over the first layer of the dielectric material; and
a micromagnet configured to exert a force on at least the first layer of dielectric material and the first layer of conductive material responsive to actuation, the force exerted by the micromagnet on at least the first layer of dielectric material and the first layer of conductive material responsive to actuation configured to applanate or indent a portion of the eye.

18. The contact lens of claim 17, wherein the sensor further comprises an application specific integrated circuit configured to measure capacitance of the sensor as the micromagnet exerts the force on at least the first layer of dielectric material and the first layer of conductive material.

19. The contact lens of claim 11, wherein the capacitance is used to determine a frequency corresponding to the strain of the lens material, the frequency being approximately 4.1395 GHz.

20. The contact lens of claim 17, wherein the sensor includes:
a second layer of dielectric material disposed over the first layer of conductive material; and
a second layer of conductive material disposed over the second layer of the dielectric material;
wherein the micromagnet is configured to exert the force on at least one of the eye, the first layer of dielectric material, the first layer of conductive material responsive to actuation, and the second layer of the dielectric material.

21. The contact lens of claim 17, wherein the sensor is responsive to a magnetic actuator configured to actuate the micromagnet.

22. The contact lens of claim 17, wherein the sensor further comprises:
a second layer of dielectric material disposed over the first layer of conductive material; and
a second layer of conductive material disposed over the second layer of the dielectric material;
an application specific integrated circuit configured to measure capacitance of the sensor as the micromagnet exerts the force on at least the first layer of dielectric material, the first layer of conductive material, and the second layer of dielectric material; and
wherein, responsive to actuation from a magnetic actuator, the micromagnet is configured to exert the force on at least the first layer of dielectric material, the first layer of conductive material responsive to actuation, and the second layer of the dielectric material.

23. The contact lens of claim 1, further comprising a tonometer system formed in the contact lens and including the sensor, and a test body in contact with the lens material configured to exert a force on an eye in the presence of a magnetic field when the contact lens is positioned on the eye.

24. The contact lens of claim 23, wherein:
the test body includes an expandable material and the sensor is in contact with the lens material;
the force exerted on the eye causes a mechanical strain in the contact lens; and
the sensor detects a rate of change of the mechanical strain of the contact lens when the force is no longer exerted on the eye.

25. The contact lens of claim 24, wherein the rate of change of the mechanical strain of the contact lens upon release of the force of the expandable material corresponds to an absolute intraocular pressure of the eye.

26. The contact lens of claim 24, wherein the test body comprises at least one of an expandable polymer material, an expandable magnetoresponsive hydrogel material, or an expandable magnetoresponsive elastomer material.

27. The contact lens of claim 24, wherein the expandable material is disposed within the lens material outside an optic zone of the contact lens.

28. The contact lens of claim 24, wherein the test body is disposed on an outer surface of the contact lens and outside and optic zone of the contact lens.

29. The contact lens of claim 24, wherein the sensor comprises a parallel plate capacitor comprising a first layer and a second layer of a conductive material and a layer of a dielectric material disposed between the first layer and the second layer of conductive material.

30. The contact lens of claim 23, wherein:
the test body includes a micromagnet disc or a micromagnet array configured to exert the force on the eye;
the sensor detects a rate of change of the mechanical strain of the contact lens when the force is no longer exerted on the eye, the rate of change of the mechanical strain of the contact lens upon release of the force of the micro-magnet corresponding to an absolute intraocular pressure of the eye.
